(19) United States
(12) Patent Application Publication
(10) Pub. No.: US 2024/0033089 A1
(43) Pub. Date: Feb. 1, 2024

(54) ADJUSTABLE ARTHROPLASTY DEVICES, SYSTEMS, AND METHODS

(71) Applicant: Optimotion Implants LLC, Orlando, FL (US)

(72) Inventors: Vuong Binh NGUYEN, Windermere, FL (US); Andrew RYNEARSON, Winter Springs, FL (US)

(73) Assignee: Optimotion Implants LLC, Orlando, FL (US)

(21) Appl. No.: 17/877,916

(22) Filed: Jul. 30, 2022

(51) Int. Cl. A61F 2/30; A61F 2/38

(52) U.S. Cl. CPC A61F 2/30734; A61F 2/38; A61F 2002/30736; A61F 2002/30738; A61F 2002/30878; A61F 2002/3054; A61F 2002/30553; A61F 2002/3069

(57) ABSTRACT

An arthroplasty system for replacing a natural articular surface on a bone may include an arthroplasty prosthesis and a bone anchoring component. The arthroplasty prosthesis may include a joint-facing side having an articular surface and a bone-facing side having a first adjustable attachment feature. The bone anchoring component may include a bone engagement surface securable to a resected surface of the bone and a second adjustable attachment feature. The first adjustable attachment feature may be configured to movably mate with the second adjustable attachment feature to movably couple the bone anchoring component to the bone-facing side of the arthroplasty prosthesis.

### TECHNICAL FIELD

The present disclosure relates to arthroplasty devices, systems, and methods. More specifically, the present disclosure relates to adjustable arthroplasty devices, systems, and methods for surgical repair of a diseased joint.

### BACKGROUND

Joint arthroplasty procedures are surgical procedures in which one or more articulating surfaces of a joint are replaced with prosthetic articulating surfaces. Such procedures are becoming increasingly commonplace, particularly for knees.

For a successful knee arthroplasty, it is important that the knee implants are correctly oriented and placed with respect to the patient's bones to remain in place, avoid subsequent complications, and maintain the necessary wear characteristics. Further, it is desirable for the knee arthroplasty procedure to be carried out quickly, smoothly, and at a lower cost. Many existing knee arthroplasty implants, systems, and methods are time-consuming to implant as they require complicated, costly, and time consuming trialing instruments and associated trialing procedures.

Accordingly, infinitely adjustable arthroplasty devices, systems, and methods that can eliminate the need for complicated, costly, and time consuming trialing instruments and procedures would be desirable.

### SUMMARY

The various arthroplasty devices, systems, and methods of the present disclosure have been developed in response to the present state of the art, and in particular, in response to the problems and needs in the art that have not yet been fully solved by currently available arthroplasty devices, systems, and methods. In some embodiments, the adjustable arthroplasty devices, systems, and methods of the present disclosure may provide improved surgical procedures and repair of a diseased joint.

In some embodiments, an arthroplasty system for replacing a natural articular surface on a bone may include an arthroplasty prosthesis and a bone anchoring component. The arthroplasty prosthesis may include a joint-facing side having an articular surface and a bone-facing side having a first adjustable attachment feature. The bone anchoring component may include a bone engagement surface securable to a resected surface of the bone and a second adjustable attachment feature. The first adjustable attachment feature may be configured to movably mate with the second adjustable attachment feature to movably couple the bone anchoring component to the bone-facing side of the arthroplasty prosthesis.

In some embodiments, the first adjustable attachment feature may include a rotary plate coupled to the bone-facing side of the arthroplasty prosthesis, and the second adjustable attachment feature may include a rotary recess formed in the bone anchoring component and configured to receive the rotary plate therein to rotatably couple the bone anchoring component to the bone-facing side of the arthroplasty prosthesis.

In some embodiments, the first adjustable attachment feature may include a rotary recess formed in the bone-facing side of the arthroplasty prosthesis, and the second adjustable attachment feature may include a rotary plate coupled to the bone anchoring component and configured to mate with the rotary recess to rotatably couple the bone anchoring component to the bone-facing side of the arthroplasty prosthesis.

In some embodiments, the first adjustable attachment feature may include a translation member coupled to the bone-facing side of the arthroplasty prosthesis, and the second adjustable attachment feature may include a translation coupler configured to movably couple with the translation member and enable the bone anchoring component to: translate along the translation member in a first direction with respect to the bone-facing side of the arthroplasty prosthesis, and translate along the translation member in a second direction, opposite the first direction, with respect to the bone-facing side of the arthroplasty prosthesis.

In some embodiments, the first adjustable attachment feature may include a translation coupler on the bone-facing side of the arthroplasty prosthesis, and the second adjustable attachment feature may include a translation member configured to movably couple with the translation coupler and enable the bone anchoring component to: translate along the translation coupler in a first direction with respect to the bone-facing side of the arthroplasty prosthesis, and translate along the translation coupler in a second direction, opposite the first direction, with respect to the bone-facing side of the arthroplasty prosthesis.

In some embodiments, the first adjustable attachment feature may include a rotary recess formed in the bone-facing side of the arthroplasty prosthesis and the second adjustable attachment feature may include a rotary plate coupled to the bone anchoring component and configured to mate with the rotary recess to rotatably couple the bone anchoring component to the bone-facing side of the arthroplasty prosthesis. The rotary plate may also include a translation member coupled to the rotary plate. The bone anchoring component may also include a translation coupler configured to movably couple with the translation member and enable the bone anchoring component to: translate along the translation member in a first direction with respect to the rotary plate and translate along the translation member in a second direction, opposite the first direction, with respect to the rotary plate.

In some embodiments, the arthroplasty prosthesis may include a tibial base plate having a tibial articular surface on the joint-facing side of the tibial base plate, and the bone anchoring component may include at least one of: a keel, a stem, a sleeve, and an adapter.

In some embodiments, the arthroplasty prosthesis may include a femoral prosthesis having a femoral articular surface on the joint-facing side of the femoral prosthesis and the bone anchoring component may include at least one of: a keel, a stem, a sleeve, and an adapter.

In some embodiments, a knee arthroplasty system for replacing a natural articular surface on a bone of a knee joint may include a knee arthroplasty prosthesis, a primary bone anchoring component, and a revision bone anchoring component. The knee arthroplasty prosthesis may include a joint-facing side having an articular surface and a bone-facing side having a prosthesis attachment feature. The primary bone anchoring component may include a primary bone engagement surface securable to a resected surface of the bone and a primary attachment feature. At least a majority of the primary bone anchoring component may be located outside an intramedullary canal of the bone when the primary bone anchoring component is anchored to the bone. The revision bone anchoring component may include a revision attachment feature and a stem having a distal portion securable within the intramedullary canal of the bone when the revision bone anchoring component is anchored to the bone. The prosthesis attachment feature may be configured to removably couple the primary bone anchoring component to the bone-facing side of the knee arthroplasty prosthesis by mating with the primary attachment feature. The prosthesis attachment feature may be configured to removably couple the revision bone anchoring component to the bone-facing side of the knee arthroplasty prosthesis by mating with the revision attachment feature.

In some embodiments, the prosthesis attachment feature may include a prosthesis plate coupled to the bone-facing side of the knee arthroplasty prosthesis. The primary attachment feature may include a primary recess formed in the primary bone anchoring component and configured to removably mate with the prosthesis plate, and the revision attachment feature may include a revision recess formed in the revision bone anchoring component and configured to removably mate with the prosthesis plate.

In some embodiments, the prosthesis attachment feature may include a prosthesis recess formed in the bone-facing side of the knee arthroplasty prosthesis. The primary attachment feature may include a primary plate coupled to the primary bone anchoring component and configured to removably mate with the prosthesis recess, and the revision attachment feature may include a revision plate coupled to the revision bone anchoring component and configured to removably mate with the prosthesis recess.

In some embodiments, the prosthesis attachment feature may include a prosthesis translation member on the bone-facing side of the knee arthroplasty prosthesis. The primary attachment feature may include a primary translation coupler configured to removably mate with the prosthesis translation member, and the revision attachment feature may include a revision translation coupler configured to removably mate with the prosthesis translation member.

In some embodiments, the prosthesis attachment feature may include a prosthesis translation coupler on the bone-facing side of the knee arthroplasty prosthesis. The primary attachment feature may include a primary translation member configured to removably mate with the prosthesis translation coupler, and the revision attachment feature may include a revision translation member configured to removably mate with the prosthesis translation coupler.

In some embodiments, the knee arthroplasty prosthesis may include a tibial base plate having a tibial articular surface on the joint-facing side of the tibial base plate, and the primary bone anchoring component may include at least one of: a short stem, a keel, a sleeve, and an adapter.

In some embodiments, the revision bone anchoring component may also include at least one of: a keel, a sleeve, and an adapter.

In some embodiments, the knee arthroplasty prosthesis may include a femoral prosthesis having a femoral articular surface on the joint-facing side of the femoral prosthesis, and the primary bone anchoring component may include at least one of: a short stem, a keel, a sleeve, and an adapter.

In some embodiments, the revision bone anchoring component may also include at least one of: a keel, a sleeve, and an adapter.

In some embodiments, a knee arthroplasty system for replacing a natural articular surface on a bone of a knee joint may include a knee arthroplasty prosthesis, a primary bone anchoring component, and a revision bone anchoring component. The primary bone anchoring component may include a joint-facing side having an articular surface and a bone-facing side having a prosthesis attachment feature formed thereon. The primary bone anchoring component may include a primary bone engagement surface securable to a resected primary surface of the bone and a primary attachment feature. The revision bone anchoring component may include a revision bone engagement surface securable to a resected revision surface of the bone and a revision attachment feature. The prosthesis attachment feature may be configured to mate with either of the primary attachment feature and the revision attachment feature to couple either of the primary bone anchoring component and the revision bone anchoring component to the bone-facing side of the knee arthroplasty prosthesis. The prosthesis attachment feature may be configured to rotatably and/or translatably mate with at least one of the primary attachment feature and the revision attachment feature.

In some embodiments, the prosthesis attachment feature may include a prosthesis plate coupled to the bone-facing side of the knee arthroplasty prosthesis. The primary attachment feature may include a primary recess formed in the primary bone anchoring component and configured to rotatably mate with the prosthesis plate, and the revision attachment feature may include a revision recess formed in the revision bone anchoring component and configured to rotatably mate with the prosthesis plate.

In some embodiments, the prosthesis attachment feature may include a prosthesis recess formed in the bone-facing side of the knee arthroplasty prosthesis. The primary attachment feature may include a primary plate coupled to the primary bone anchoring component and configured to rotatably mate with the prosthesis recess, and the revision attachment feature may include a revision plate coupled to the revision bone anchoring component and configured to rotatably mate with the prosthesis recess.

In some embodiments, the prosthesis attachment feature may include a prosthesis translation member on the bone-facing side of the knee arthroplasty prosthesis. The primary attachment feature may include a primary translation coupler configured to translatably mate with the prosthesis translation member, and the revision attachment feature may include a revision translation coupler configured to translatably mate with the prosthesis translation member.

In some embodiments, the prosthesis attachment feature may include a prosthesis translation coupler on the bone-facing side of the knee arthroplasty prosthesis. The primary attachment feature may include a primary translation member configured to translatably mate with the prosthesis translation coupler, and the revision attachment feature may include a revision translation member configured to translatably mate with the prosthesis translation coupler.

In some embodiments, the prosthesis attachment feature may include a prosthesis recess formed in the bone-facing side of the knee arthroplasty prosthesis and the primary attachment feature may include a primary plate coupled to the primary bone anchoring component and configured to rotatably mate with the prosthesis recess to rotatably couple the primary bone anchoring component to the bone-facing side of the knee arthroplasty prosthesis. The primary plate may also include a primary translation member coupled to the primary plate. The primary bone anchoring component may also include a primary translation coupler configured to translatably mate with the primary translation member and enable the primary bone anchoring component to: translate along the primary translation member in a first direction with respect to the primary plate and translate along the primary translation member in a second direction, opposite the first direction, with respect to the primary plate.

In some embodiments, the prosthesis attachment feature may include a prosthesis recess formed in the bone-facing side of the knee arthroplasty prosthesis and the revision attachment feature may include a revision plate coupled to the revision bone anchoring component and configured to rotatably mate with the prosthesis recess to rotatably couple the revision bone anchoring component to the bone-facing side of the knee arthroplasty prosthesis. The revision plate may also include a revision translation member coupled to the revision plate. The revision bone anchoring component may also include a revision translation coupler configured to translatably mate with the revision translation member and enable the revision bone anchoring component to: translate along the revision translation member in a first direction with respect to the revision plate and translate along the revision translation member in a second direction, opposite the first direction, with respect to the revision plate.

In some embodiments, the knee arthroplasty prosthesis may include a tibial base plate having a tibial articular surface on the joint-facing side of the tibial base plate.

In some embodiments, the knee arthroplasty prosthesis may include a femoral prosthesis having a femoral articular surface on the joint-facing side of the femoral prosthesis.

In some embodiments, the primary bone anchoring component may include at least one of: a short stem, a keel, a sleeve, and an adapter.

In some embodiments, at least a majority of the primary bone anchoring component may be located outside an intramedullary canal of the bone when the primary bone anchoring component is anchored to the bone.

In some embodiments, the revision bone anchoring component may include a stem. A distal portion of the stem may be securable within an intramedullary canal of the bone when the revision bone anchoring component is anchored to the bone.

In some embodiments, the revision bone anchoring component may also include at least one of: a keel, a sleeve, and an adapter.

In some embodiments, an arthroplasty system for replacing a natural articular surface on a bone may include an arthroplasty prosthesis having a joint-facing side having an articular surface and a bone-facing side having a prosthesis attachment feature. The arthroplasty system may also include a bone anchoring component having a bone engagement surface securable to a resected surface of the bone and a component attachment feature. The component attachment feature may be configured to removably mate with the prosthesis attachment feature to removably couple the bone anchoring component to the bone-facing side of the arthroplasty prosthesis, and the prosthesis attachment feature may include a circular shape having a diameter of at least 10 mm.

In some embodiments, the prosthesis attachment feature may include a first circular shape having a first diameter of at least 10 mm, the component attachment feature may include a second circular shape having a second diameter of at least 10 mm, and the prosthesis attachment feature and the component attachment feature may include complementary circular shapes with each other.

In some embodiments, the diameter of the circular shape may be at least 20 mm.

In some embodiments, the diameter of the circular shape may be at least 30 mm.

In some embodiments, the prosthesis attachment feature may include a prosthesis plate coupled to the bone-facing side of the arthroplasty prosthesis, and the component attachment feature may include a component recess configured to removably mate with the prosthesis plate to removably couple the bone anchoring component to the bone-facing side of the arthroplasty prosthesis.

In some embodiments, the prosthesis attachment feature may include a prosthesis recess formed in the bone-facing side of the arthroplasty prosthesis, and the component attachment feature may include a component plate coupled to the bone anchoring component and configured to removably mate with the prosthesis recess to removably couple the bone anchoring component to the bone-facing side of the arthroplasty prosthesis.

In some embodiments, the arthroplasty prosthesis may include a femoral prosthesis having a femoral articular surface on the joint-facing side of the femoral prosthesis.

In some embodiments, the arthroplasty prosthesis may include a tibial base plate having a tibial articular surface on the joint-facing side of the tibial base plate.

In some embodiments, the tibial base plate may include at least one first anti-rotation feature formed on the bone-facing side of the tibial base plate, and the bone anchoring component may include at least one second anti-rotation feature configured to removably mate with the first anti-rotation feature and resist rotation of the bone anchoring component with respect to the tibial base plate.

In some embodiments, the bone anchoring component may include at least one of: a keel, a stem, a sleeve, and an adapter.

These and other features and advantages of the present disclosure will become more fully apparent from the following description and appended claims or may be learned by the practice of the arthroplasty devices, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1 illustrates an exploded view of an arthroplasty system, according to an embodiment of the present disclosure;

FIG. 2 illustrates a front perspective view of the arthroplasty system of FIG. 1, after assembly;

FIG. 3 illustrates a rear perspective view of the arthroplasty system of FIG. 2;

FIG. 4A illustrates a top perspective view of a tibial base plate, according to an embodiment of the present disclosure;

FIG. 4B illustrates a bottom perspective view of the tibial base plate of FIG. 4A;

FIG. 4C illustrates a top view of the tibial base plate of FIG. 4A;

FIG. 4D illustrates a bottom view of the tibial base plate of FIG. 4A;

FIG. 5A illustrates a front view of a keel, according to an embodiment of the present disclosure;

FIG. 5B illustrates a side view of the keel of FIG. 5A;

FIG. 5C illustrates a top view of the keel of FIG. 5A;

FIG. 5D illustrates a bottom view of the keel of FIG. 5A;

FIG. 6 illustrates an exploded view of an arthroplasty system, according to another embodiment of the present disclosure;

FIG. 7 illustrates a front perspective view of the arthroplasty system of FIG. 6, after assembly;

FIG. 8 illustrates a rear perspective view of the arthroplasty system of FIG. 7;

FIG. 9A illustrates a front view of a low profile keel, according to another embodiment of the present disclosure;

FIG. 9B illustrates a side view of the low profile keel of FIG. 9A;

FIG. 9C illustrates a top view of the low profile keel of FIG. 9A;

FIG. 9D illustrates a bottom view of the low profile keel of FIG. 9A;

FIG. 10 illustrates an exploded view of an arthroplasty system, according to another embodiment of the present disclosure;

FIG. 11 illustrates a perspective view of the arthroplasty system of FIG. 10, after assembly;

FIG. 12 illustrates a perspective view of the arthroplasty system of FIG. 11;

FIG. 13 illustrates a cross-sectional view of the arthroplasty system of FIG. 12;

FIG. 14A illustrates a bottom perspective view of a component attachment feature, according to an embodiment of the present disclosure;

FIG. 14B illustrates a side view of the component attachment feature of FIG. 14A;

FIG. 14C illustrates a top view of the component attachment feature of FIG. 14A;

FIG. 14D illustrates a bottom view of the component attachment feature of FIG. 14A;

FIG. 15A illustrates a perspective view of a stem adapter, according to an embodiment of the present disclosure;

FIG. 15B illustrates another perspective view of the stem adapter of FIG. 15A;

FIG. 15C illustrates a top view of the stem adapter of FIG. 15A;

FIG. 15D illustrates a side view of the stem adapter of FIG. 15A;

FIG. 16 illustrates an exploded view of an arthroplasty system, according to another embodiment of the present disclosure;

FIG. 17 illustrates a bottom perspective view of the arthroplasty system of FIG. 16, after assembly;

FIG. 18 illustrates a front view of the arthroplasty system of FIG. 17;

FIG. 19 illustrates a bottom view of the arthroplasty system of FIG. 17;

FIG. 20 illustrates an exploded view of an arthroplasty system, according to another embodiment of the present disclosure;

FIG. 21 illustrates a perspective view of the arthroplasty system of FIG. 20, after assembly;

FIG. 22A illustrates a perspective view of a cone-shaped sleeve, according to an embodiment of the present disclosure;

FIG. 22B illustrates a side view of the cone-shaped sleeve of FIG. 22A;

FIG. 22C illustrates a top view of the cone-shaped sleeve of FIG. 22A;

FIG. 22D illustrates a bottom view of the cone-shaped sleeve of FIG. 22A;

FIG. 23 illustrates an exploded view of an arthroplasty system, according to another embodiment of the present disclosure;

FIG. 24 illustrates a perspective view of the arthroplasty system of FIG. 23, after assembly;

FIG. 25A illustrates a perspective view of an oblong sleeve, according to another embodiment of the present disclosure;

FIG. 25B illustrates a side view of the oblong sleeve of FIG. 25A;

FIG. 25C illustrates a top view of the oblong sleeve of FIG. 25A;

FIG. 25D illustrates a bottom view of the oblong sleeve of FIG. 25A;

FIG. 26A illustrates a perspective view of a broach, according to an embodiment of the present disclosure;

FIG. 26B illustrates another perspective view of the broach of FIG. 26A;

FIG. 26C illustrates a side view of the broach of FIG. 26A;

FIG. 26D illustrates a bottom view of the broach of FIG. 26A;

FIG. 27 illustrates an exploded view of an arthroplasty system, according to another embodiment of the present disclosure;

FIG. 28 illustrates a perspective view of the arthroplasty system of FIG. 27, after assembly;

FIG. 29A illustrates a lateral side view of a femoral prosthesis, according to an embodiment of the present disclosure;

FIG. 29B illustrates a rear view of the femoral prosthesis of FIG. 29A;

FIG. 29C illustrates a medial side view of the femoral prosthesis of FIG. 29A;

FIG. 29D illustrates a front view of the femoral prosthesis of FIG. 29A;

FIG. 29E illustrates a top view of the femoral prosthesis of FIG. 29A;

FIG. 29F illustrates a bottom view of the femoral prosthesis of FIG. 29A;

FIG. 30 illustrates an exploded view of an arthroplasty system, according to another embodiment of the present disclosure; and

FIG. 31 illustrates a perspective view of the arthroplasty system of FIG. 30, after assembly.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

As defined herein, the terms “arthroplasty prosthesis” and “arthroplasty system” (and their analogs) encompasses any prosthesis or prosthesis system comprising a bone-facing side that can be implanted within, adjacent, or near a bone of a patient or an animal. Thus, the terms “arthroplasty prosthesis” and “arthroplasty system” can also include prostheses or prostheses systems that may or may not be utilized to repair a diseased joint of a patient or an animal.

As defined herein, the term “bone anchoring component” (and its analogs) encompasses any prosthesis that comprises at least one bone-engaging surface (or at least one bone cement engaging surface, or at least one bone augment material engaging surface, etc.), or that is coupled to or supports (either directly or indirectly) another component of the prosthesis that comprises the at least one bone-engaging surface (or the at least one bone cement engaging surface, or the at least one bone augment material engaging surface, etc.) that is implantable within, adjacent, or near a bone of a patient or an animal. Accordingly, the term the term “bone anchoring component” can include any a stem (e.g., short stems, long stems, intermediate stems, etc.), intramedullary rod, keel, sleeve, adapter, augment, etc., that has any size, shape, feature, morphology, etc., that is described or contemplated herein, as well as any a stem (short stems, long stems, etc.), intramedullary rod, keel, sleeve, adapter, augment, etc., that has any size, shape, feature, morphology, etc., that is not described or contemplated herein.

Although the arthroplasty prostheses and systems described herein are disclosed in the context of knee arthroplasty prostheses and systems, it will be readily understood by those of skill in the art that the inventive concepts described and contemplated herein may be applied to any prosthesis or prosthesis system for any bone or joint of a patient or an animal, including, but not limited to, bones/joints of the foot, ankle, knee, hip, hand, wrist, elbow, shoulder, sacral, spine, neck, etc., without departing from the spirit or scope of the present disclosure.

It will also be understood that any component or feature of any arthroplasty system described or contemplated herein may be combined with any other component or feature that is described or contemplated herein to create any number of different arthroplasty system embodiments.

It will also be understood that any component or feature of any arthroplasty system described or contemplated herein may be combined with any other component or feature that is described or contemplated herein to create any number of different surgical kits. Each of these surgical kits may also include (or not include) any number of supporting surgical instruments or general parts (e.g., bone screws, fasteners, K-wires, etc.).

Moreover, it will also be understood that any method step (or component/feature of any method step) that is described or contemplated herein may be combined with any other method step (or component/feature of any method step) that is described or contemplated herein in any order, and/or in any combination, to create any number of different method embodiments for implanting any number of different arthroplasty system embodiments that are described or contemplated herein.

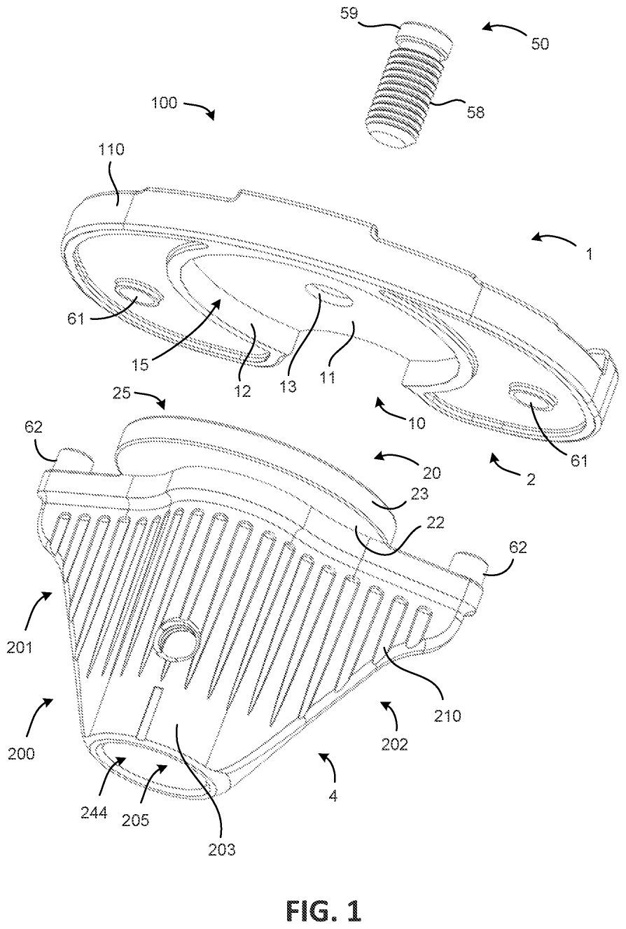

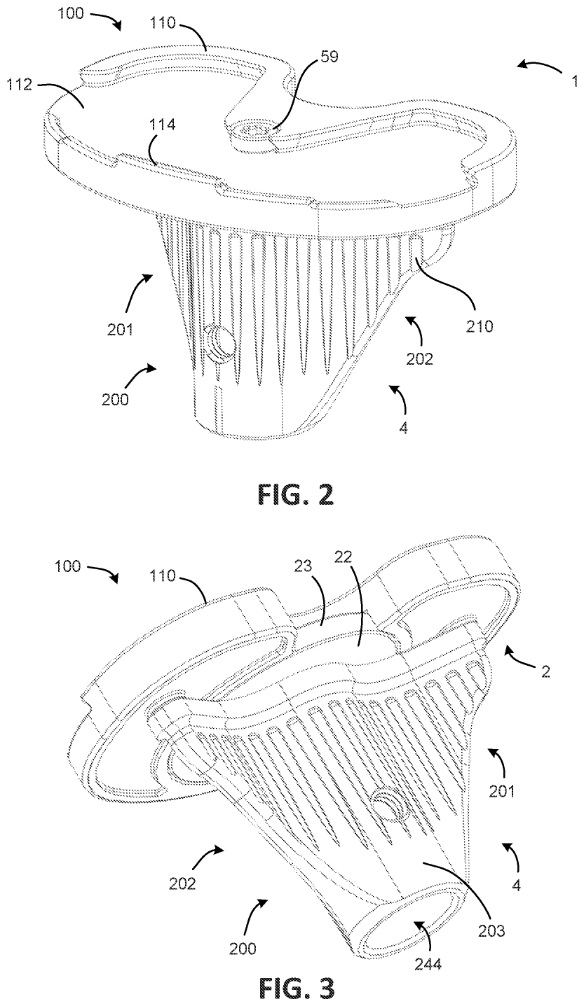

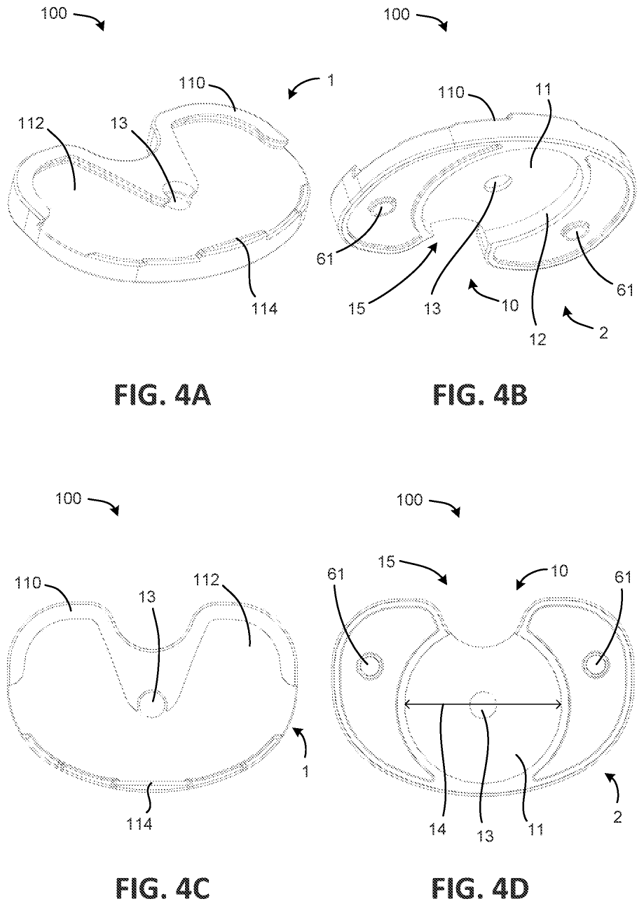

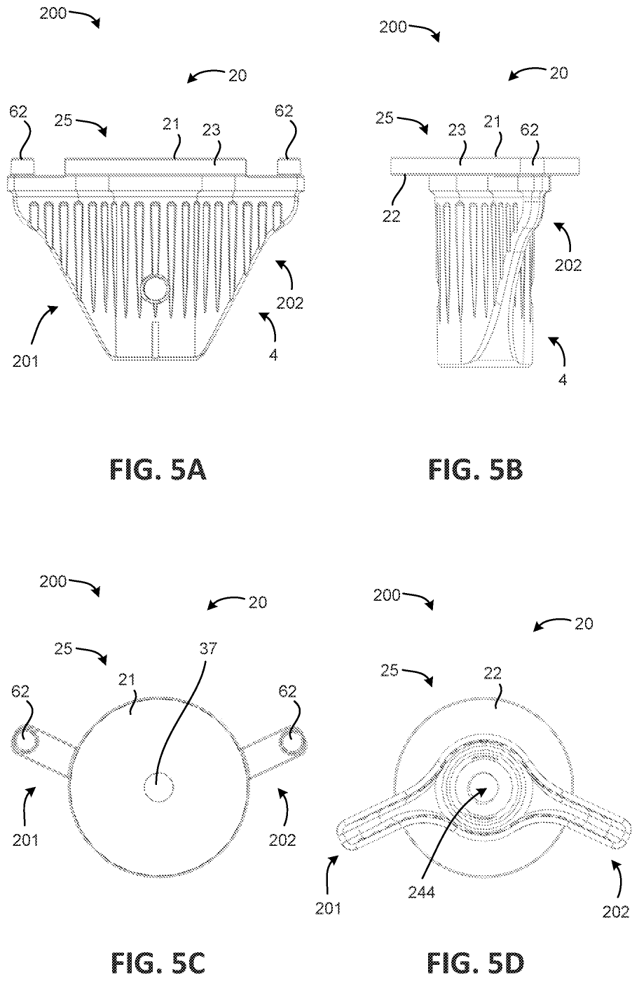

FIGS. 1-5D illustrate various views of an arthroplasty system, according to an embodiment of the present disclosure. Specifically, FIG. 1 is an exploded view of the arthroplasty system; FIG. 2 is a front perspective view of the arthroplasty system, after assembly; FIG. 3 is a rear perspective view of the arthroplasty system; FIG. 4A is a top perspective view of a tibial base plate of the arthroplasty system; FIG. 4B is a bottom perspective view of the tibial base plate; FIG. 4C is a top view of the tibial base plate; FIG. 4D is a bottom view of the tibial base plate; FIG. 5A is a front view of a keel of the arthroplasty system; FIG. 5B is a side view of the keel; FIG. 5C is a top view of the keel; and FIG. 5D is a bottom view of the keel.

In some embodiments, the arthroplasty system of FIGS. 1-5D may generally include a tibial base plate 100, a keel 200, and a locking fastener 50.

In some embodiments, the locking fastener 50 may include a locking fastener torque connection interface 59 and a locking fastener thread 58.

In some embodiments, the tibial base plate 100 may be a universal tibial base plate that may be compatible with some or all of the other bone anchoring components that are described or contemplated herein.

The tibial base plate 100 may also be referred to herein as an arthroplasty prosthesis or a knee arthroplasty prosthesis. The tibial base plate 100 may generally include a joint-facing side 1, a bone-facing side 2, a peripheral ridge 110, an interior recess 112, an alcove 114, and at least one first anti-rotation feature 61 (or first anti-rotation hole/first augment hole).

In some embodiments, the joint-facing side 1 of the tibial base plate 100 may include a tibial articular surface, such as a tibial articular surface of a tibial insert (not shown) that may be couplable with the interior recess 112 of the tibial base plate 100 and held in place by the alcove 114 and/or the peripheral ridge 110 of the tibial base plate 100. However, in other embodiments, the joint-facing side 1 of the tibial base plate 100 may directly incorporate a tibial articular surface (not shown) that may be integrally formed with the tibial base plate 100.

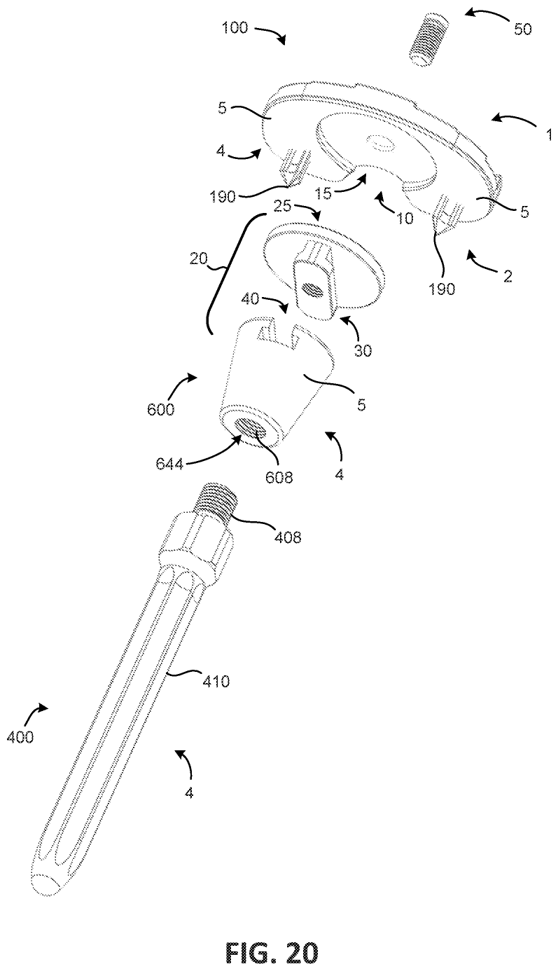

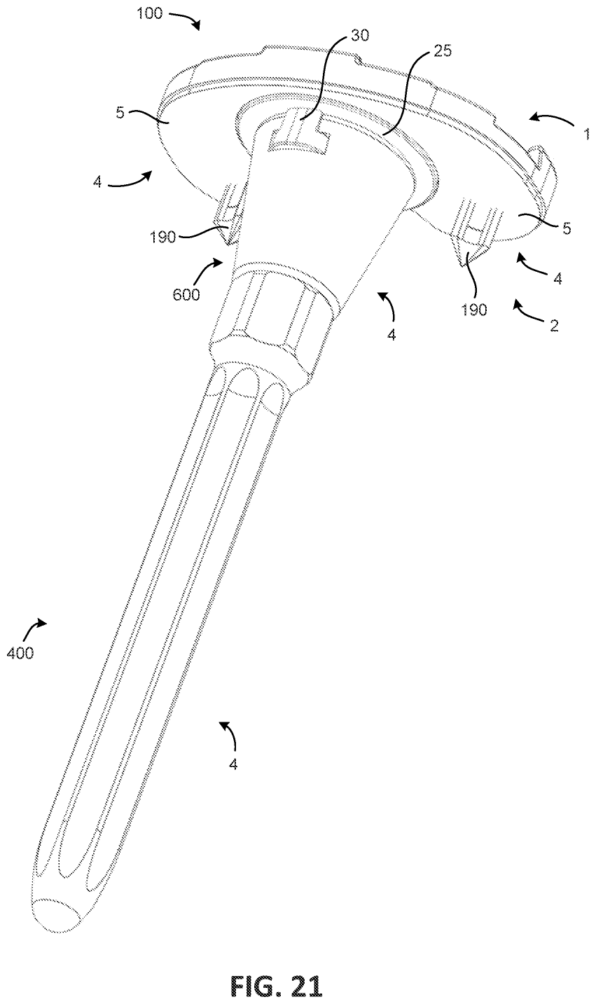

In some embodiments, the bone-facing side 2 of the tibial base plate 100 may include a bone engagement surface 4 (e.g., see FIGS. 20 and 21). The bone engagement surface 4 may also be referred to herein as a primary bone engagement surface or a revision bone engagement surface. In some embodiments, the bone engagement surface 4 of the tibial base plate 100 may include a porous surface coating 5 (see FIGS. 20 and 21). In some embodiments, the porous surface coating 5 may be an additive surface coating (e.g., a porous titanium surface, or other porous metal surface), a nano-coating, or other treatment that may be configured to promote bone ingrowth for cementless (or cemented) applications.

In some embodiments, the bone-facing side 2 of the tibial base plate 100 may not include a bone engagement surface.

In some embodiments, the bone-facing side 2 of the tibial base plate 100 may be intended for use with a bone cement, a bone augment material, etc.

In some embodiments, the bone-facing side 2 of the tibial base plate 100 may include at least one post 190 (e.g., see FIGS. 20 and 21) that may project inferiorly away from the bone-facing side 2 of the tibial base plate 100.

As shown in FIG. 1, in some embodiments the tibial base plate 100 may include a prosthesis attachment feature 10 or first adjustable attachment feature on the bone-facing side 2 of the tibial base plate 100.

In some embodiments, the prosthesis attachment feature 10 may be formed in/on the bone-facing side 2 of the tibial base plate 100. The prosthesis attachment feature 10 may include a rotary recess 15 having a recess surface 11, a recess sidewall 12, a recess fastener hole 13, and a recess diameter 14. The rotary recess 15 may also be referred to herein as a prosthesis recess, a component recess, a primary recess, or a revision recess. However, In some embodiments, the prosthesis attachment feature 10 of the tibial base plate 100 may include a rotary plate, as will be discussed in more detail below.

Continuing with FIGS. 1-5D, the keel 200 may also be referred to herein as a bone anchoring component, a primary bone anchoring component, or a revision bone anchoring component.

In some embodiments, the keel 200 may generally include a first keel web 201, a second keel web 202, a central keel post 203, a keel bore 244, a keel bore thread 205, and one or more keel ribs 210 or crenellations formed in the bone engagement surface 4 of the keel 200.

In some embodiments, the keel 200 may also include at least one second anti-rotation feature 62 and a component attachment feature 20 that may be coupled to, or formed in/on, the proximal end of the keel 200.

In some embodiments, the at least one second anti-rotation feature 62 may be tapered and received within the at least one first anti-rotation feature 61 formed in the tibial base plate 100 to form a tapered connection that may prevent relative rotation of the keel 200 and the tibial base plate 100, as will be discussed below in more detail.

The component attachment feature 20 may also be referred to herein as a second adjustable attachment feature, a primary attachment feature, or a revision attachment feature.

In some embodiments, the component attachment feature 20 may comprise a rotary plate 25 having a plate diameter 24, a prosthesis-facing surface 21, a component-facing surface 22, and a plate sidewall 23.

However, in other embodiments the structures of the component attachment feature 20 and the prosthesis attachment feature 10 may be reversed, such that the component attachment feature 20 may comprise the rotary recess 15 and the prosthesis attachment feature 10 may comprise the rotary plate 25. Thus, in some embodiments the prosthesis attachment feature 10 and the component attachment feature 20 may comprise complementary circular shapes with each other.

The rotary plate 25 may also be referred to herein as a prosthesis plate, a component plate, a primary plate, or a revision plate.

In some embodiments, the prosthesis attachment feature 10 may comprise a circular shape having a diameter of at least 10 mm.

However, in some embodiments the prosthesis attachment feature 10 may also comprise a circular shape having a diameter that is less than 10 mm.

In some embodiments, the prosthesis attachment feature 10 may comprise a circular shape having a diameter of at least 15 mm.

In some embodiments, the prosthesis attachment feature 10 may comprise a circular shape having a diameter of at least 20 mm.

In some embodiments, the prosthesis attachment feature 10 may comprise a circular shape having a diameter of at least 25 mm.

In some embodiments, the prosthesis attachment feature 10 may comprise a circular shape having a diameter of at least 30 mm.

In some embodiments, the prosthesis attachment feature 10 may comprise a circular shape having a diameter of at least 35 mm.

In some embodiments, the prosthesis attachment feature 10 may comprise a circular shape having a diameter of at least 40 mm.

In some embodiments, the prosthesis attachment feature 10 may comprise a circular shape having a diameter of at least 45 mm.

In some embodiments, the prosthesis attachment feature 10 may comprise a circular shape having a diameter of at least 50 mm.

In some embodiments, the prosthesis attachment feature 10 may comprise a circular shape having a diameter of at least 55 mm.

In some embodiments, the prosthesis attachment feature 10 may comprise a circular shape having a diameter of at least 60 mm.

In some embodiments, the prosthesis attachment feature 10 may comprise a circular shape having a diameter of at least 65 mm.

In some embodiments, the prosthesis attachment feature 10 may comprise a circular shape having a diameter of at least 70 mm.

In some embodiments, the prosthesis attachment feature 10 may comprise a circular shape having a diameter of at least 75 mm.

In some embodiments, the prosthesis attachment feature 10 may comprise a circular shape having a diameter that is at least 10% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 15% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 20% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 25% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 30% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 35% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 40% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 45% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 50% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 55% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 60% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 65% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 70% of the medial-lateral width or anterior-posterior width of the arthroplasty prosthesis comprising the prosthesis attachment feature 10.

In some embodiments, the prosthesis attachment feature 10 may comprise a circular shape having a diameter that is at least 75% of the medial-lateral width or anterior-posterior width of the arthroplasty prosthesis comprising the prosthesis attachment feature 10.

As can be seen in FIGS. 1-5D, in some embodiments the component attachment feature 20 may be configured to removably mate with the prosthesis attachment feature 10 to removably couple the bone anchoring component to the bone-facing side 2 of the arthroplasty prosthesis.

In some embodiments, the first adjustable attachment feature or prosthesis attachment feature 10 may include the rotary recess 15 formed in the bone-facing side 2 of the arthroplasty prosthesis, and the second adjustable attachment feature or component attachment feature 20 may include the rotary plate 25 coupled to the bone anchoring component and configured to removably mate with the rotary recess 15. In these embodiments, the keel 200 may not move/rotate with respect to the tibial base plate 100, given the at least one second anti-rotation feature 62 of the keel 200 that may be configured to mate with the at least one first anti-rotation feature 61 of the tibial base plate 100 to prevent relative rotation between the keel 200 and the tibial base plate 100 after assembly with the locking fastener 50 coupling the tibial base plate 100 to the keel 200. However, it will also be understood that some embodiments may utilize alternative locking/anti-rotation features in place of, or in addition to, the first and second anti-rotation features 61, 62 to lock the keel 200 to the tibial base plate 100 after assembly and/or prevent relative rotation between the keel 200 and the tibial base plate 100. For example, in some embodiments at least one of the recess surface 11 and the recess sidewall 12 may include a first surface feature (not shown), and at least one of the prosthesis-facing surface 21 and the plate sidewall 23 may include a second surface feature (not shown). The first and second surface features may be shaped and/or configured to interface with each other to lock the keel 200 to the tibial base plate 100 after assembly and/or prevent relative rotation between the keel 200 and the tibial base plate 100. Example surface features that may be utilized to achieve such locking/anti-rotation functionality may include, but are not limited to: one or more jagged surfaces/edges, one or more roughened surfaces/edges, one or more projections formed on at least one of the surfaces/edges, one or more indentations formed in at least one of the surfaces/edges, etc.

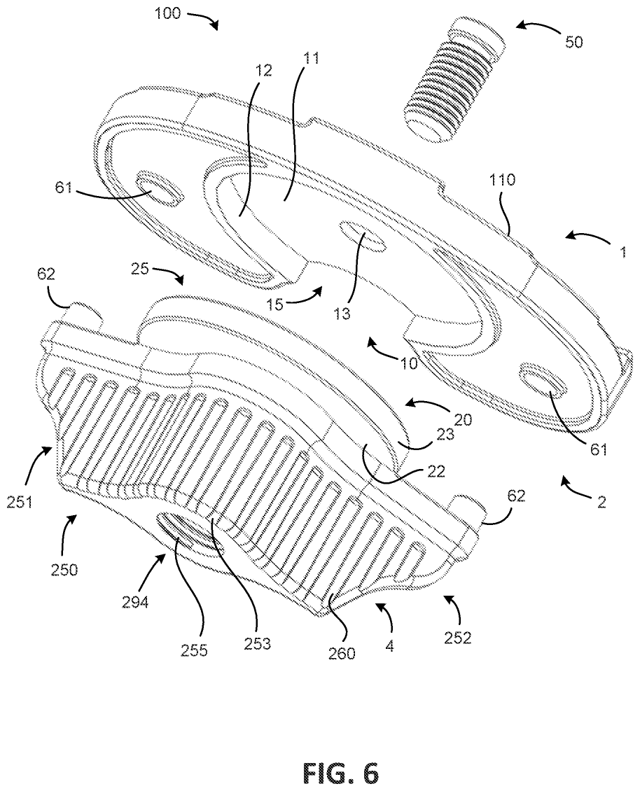

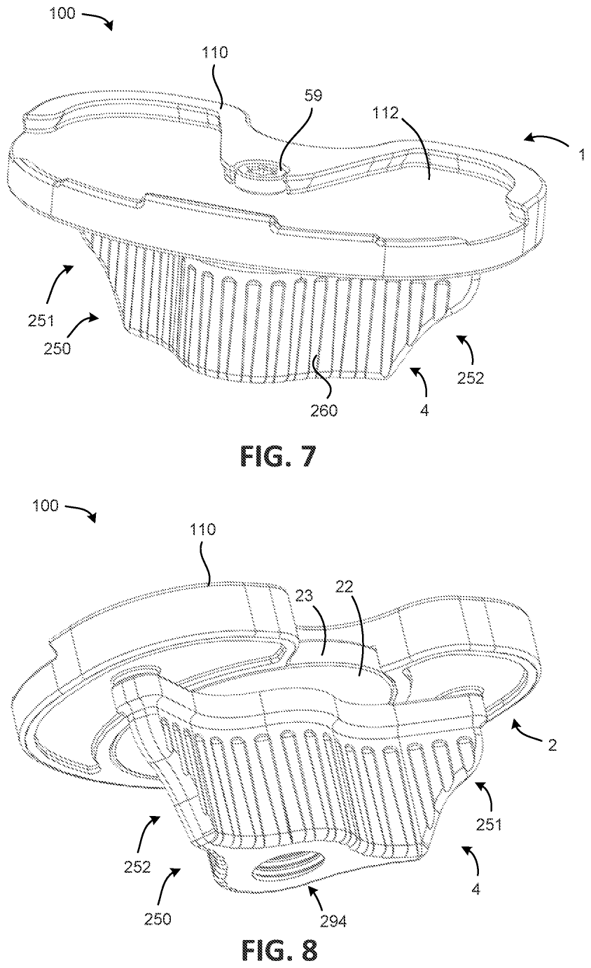

In some embodiments, the arthroplasty system of FIGS. 1-5D may be utilized for primary knee arthroplasty procedures that do not require a stem or intramedullary rod for extra stability. However, it will also be understood that the arthroplasty system of FIGS. 6-9D may also be utilized for revision knee arthroplasty procedures as well.

In some embodiments, the first adjustable attachment feature or prosthesis attachment feature 10 may be configured to movably mate with the second adjustable attachment feature or component attachment feature 20 to movably couple a bone anchoring component, such as the keel 200, to the bone-facing side 2 of the arthroplasty prosthesis, such as the tibial base plate 100.

In some embodiments, the first adjustable attachment feature may include the rotary recess 15 formed in the bone-facing side 2 of the arthroplasty prosthesis, and the second adjustable attachment feature may include the rotary plate 25 coupled to the bone anchoring component and configured to mate with the rotary recess 15 to rotatably couple the bone anchoring component to the bone-facing side 2 of the arthroplasty prosthesis. For example, the at least one first and/or second anti-rotation features may be omitted to allow rotatable coupling between the keel 200 and the tibial base plate 100.

In some embodiments, the first adjustable attachment feature may include the rotary plate 25 coupled to the bone-facing side 2 of the arthroplasty prosthesis, and the second adjustable attachment feature may include the rotary recess 15 formed in the bone anchoring component and configured to receive the rotary plate 25 therein to rotatably couple the bone anchoring component to the bone-facing side 2 of the arthroplasty prosthesis.

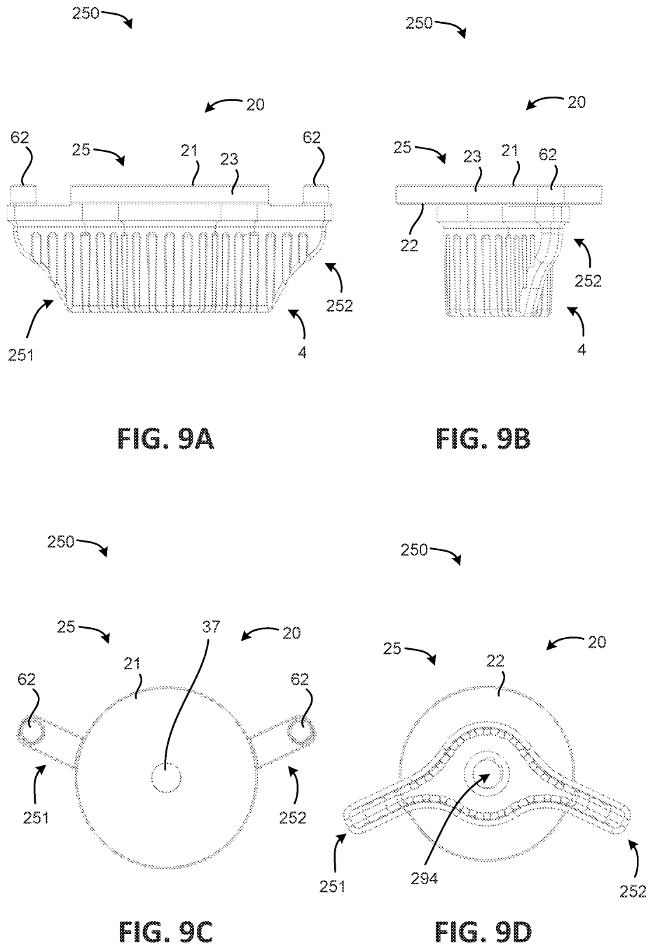

FIGS. 6-9D illustrate various views of an arthroplasty system or knee arthroplasty system, according to another embodiment of the present disclosure. Specifically, FIG. 6 is an exploded view of the arthroplasty system; FIG. 7 is a front perspective view of the arthroplasty system, after assembly; FIG. 8 is a rear perspective view of the arthroplasty system; FIG. 9A is a front view of a low profile keel of the arthroplasty system; FIG. 9B is a side view of the low profile keel; FIG. 9C is a top view of the low profile keel; and FIG. 9D is a bottom view of the low profile keel.

In some embodiments, the arthroplasty system of FIGS. 6-9D may generally include the locking fastener 50, the tibial base plate 100, and a low profile keel 250.

In some embodiments, the locking fastener 50 and the tibial base plate 100 may comprise the same/similar features to other locking fasteners and tibial base plates discussed herein.

In some embodiments, the low profile keel 250 may include similar features to the keel 200 but may have a shorter height.

In some embodiments, the low profile keel 250 may generally include a bone engagement surface 4, a first low profile keel web 251, a second low profile keel web 252, a low profile central keel post 253, a low profile keel bore 294, a keel bore thread 255, the at least one second anti-rotation feature 62, the component attachment feature 20, and one or more low profile keel ribs 260 or crenellations formed in the bone engagement surface 4.

In some embodiments, the arthroplasty system of FIGS. 6-9D may be utilized for primary knee arthroplasty procedures that do not require a stem or intramedullary rod for extra stability. However, it will also be understood that the arthroplasty system of FIGS. 6-9D may also be utilized for revision knee arthroplasty procedures as well.

In some embodiments, the knee arthroplasty system of FIGS. 6-9D may be implanted within a knee joint from a lateral approach utilizing a minimally invasive surgical procedure. The lower overall height of the low profile keel 250 may help facilitate surgical procedures utilizing a lateral approach. In some embodiments, the low profile keel 250 may be implanted in a prepared tibia from the lateral approach first, and then the tibial base plate 100 may be inserted into the surgical site from the lateral approach and coupled to the installed low profile keel 250. However, it will be understood that any of the arthroplasty systems described or contemplated herein may be implanted within any joint or bone from an anterior direction, a posterior direction, a lateral direction, a medial direction, an inferior direction, a superior direction, etc., and/or any combination of directions thereof (e.g., a postero-lateral direction, an antero-medial direction, etc.).

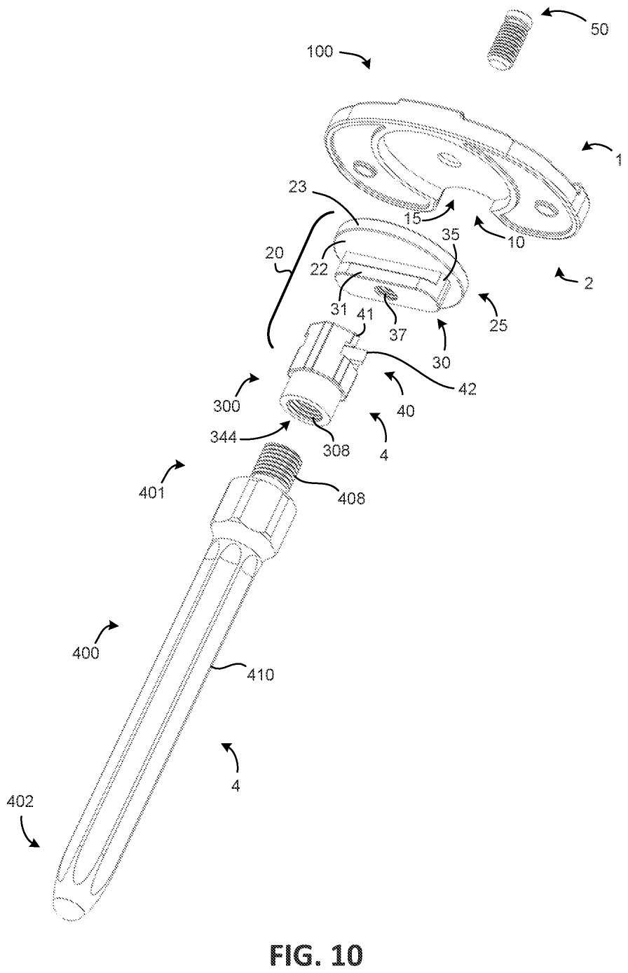

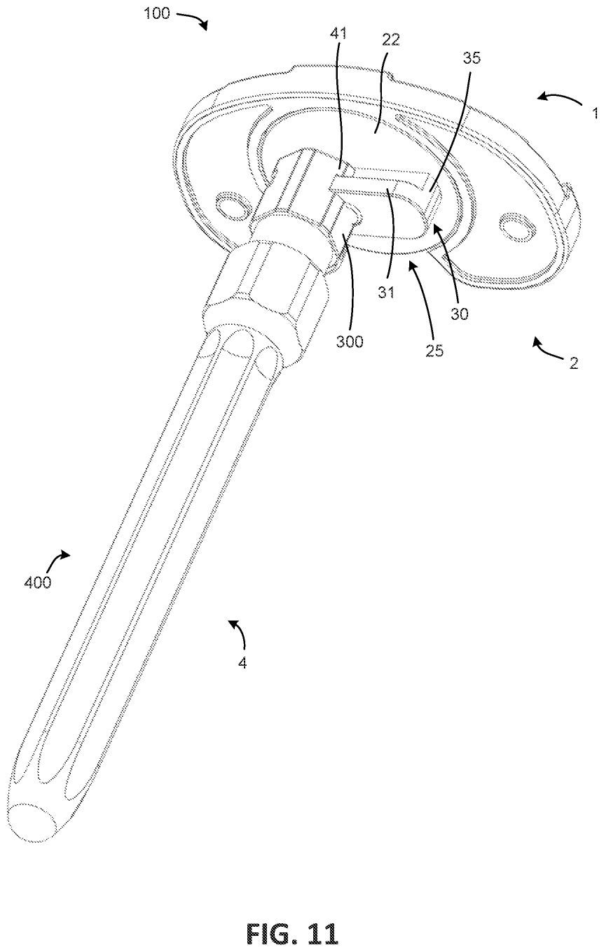

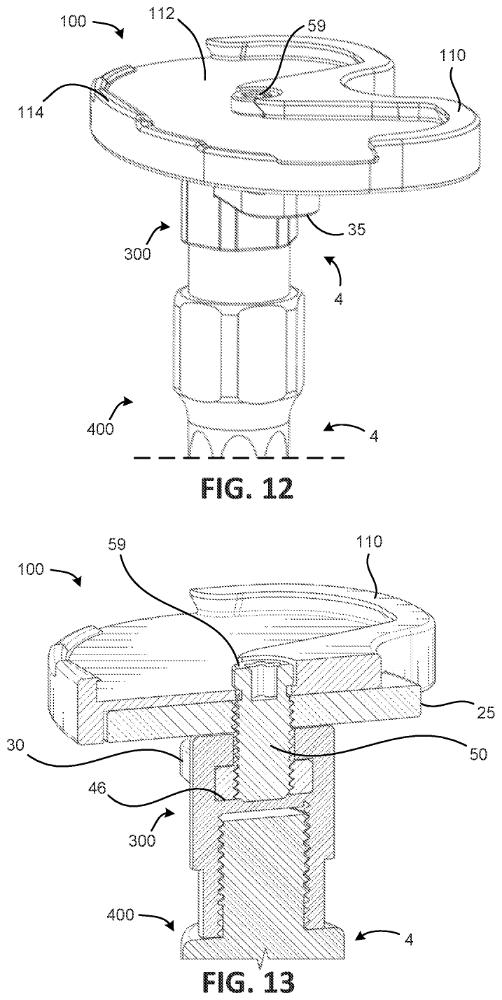

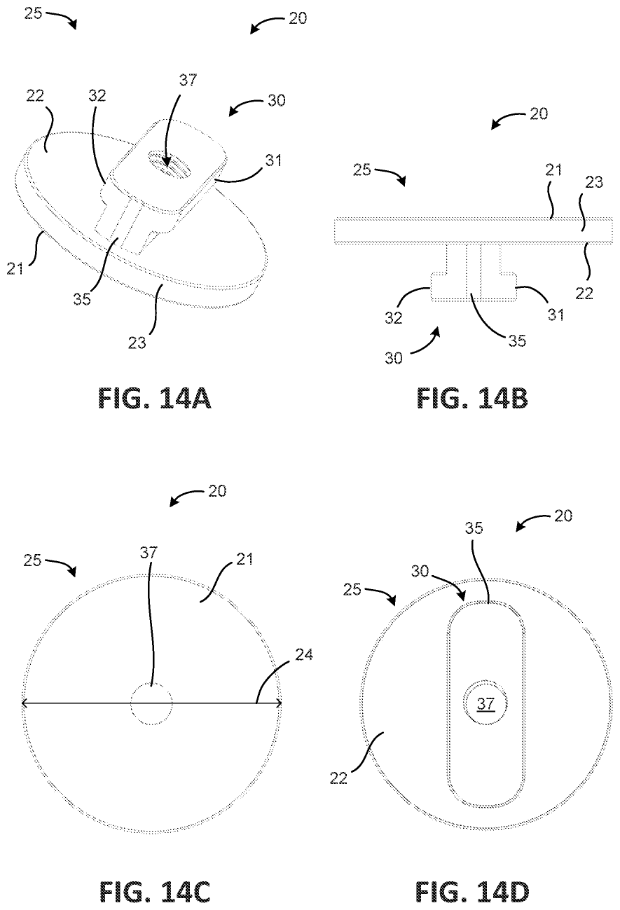

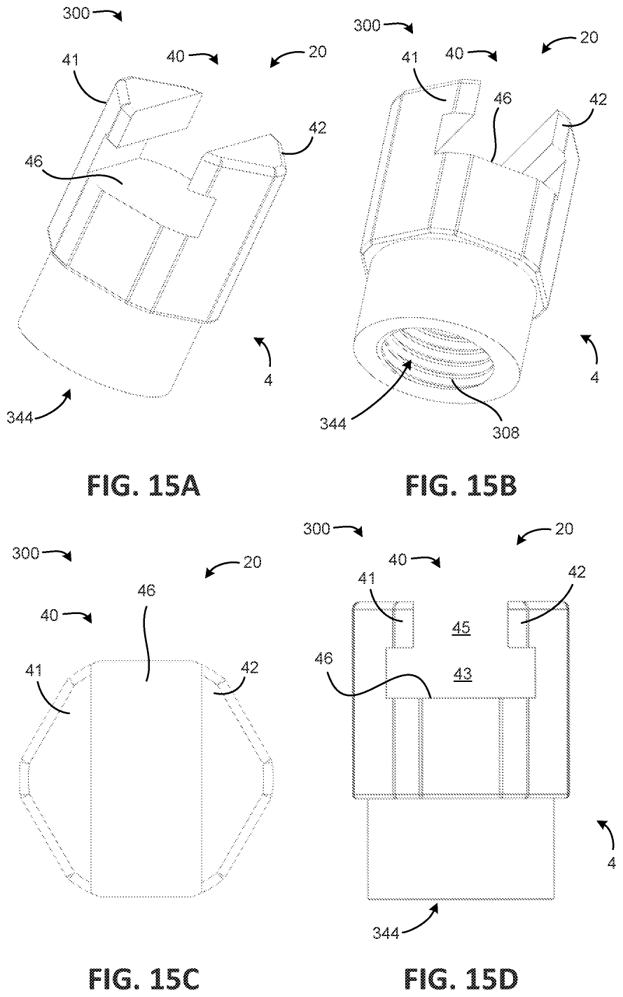

FIGS. 10-15D illustrate various views of an arthroplasty system or knee arthroplasty system, according to another embodiment of the present disclosure. Specifically, FIG. 10 is an exploded view of the arthroplasty system; FIG. 11 is a perspective view of the arthroplasty system, after assembly; FIG. 12 is another perspective view of the arthroplasty system; FIG. 13 is a cross-sectional view of the arthroplasty system of FIG. 12; FIG. 14A is a bottom perspective view of a component attachment feature of the arthroplasty system; FIG. 14B is a side view of the component attachment feature; FIG. 14C is a top view of the component attachment feature; FIG. 14D is a bottom view of the component attachment feature; FIG. 15A is a perspective view of a stem adapter of the arthroplasty system; FIG. 15B is another perspective view of the stem adapter; FIG. 15C is a top view of the stem adapter; and FIG. 15D is a side view of the stem adapter.

In some embodiments, the arthroplasty system of FIGS. 10-15D may generally include the locking fastener 50, the tibial base plate 100, the component attachment feature 20 (which may form one or more separate pieces in some embodiments), a stem adapter 300 or bone anchoring component/adapter, and a stem 400.

In some embodiments, the locking fastener 50 and the tibial base plate 100 may comprise the same/similar features to other locking fasteners and tibial base plates discussed herein.

In some embodiments, the stem 400 may generally include a proximal portion 401 including a stem thread 408, a distal portion 402, and a bone engagement surface 4 that may or may not include one or more splines 410 or ribs and may or may not have a porous surface coating.

In some embodiments, the arthroplasty system of FIGS. 10-15D may be utilized for revision knee arthroplasty procedures that may require the stem 400 or an intramedullary rod for extra stability. However, it will also be understood that the arthroplasty system of FIGS. 10-15D may also be utilized for primary knee arthroplasty procedures as well.

In some embodiments, the component attachment feature 20 may include the rotary plate 25, a translation member 30 that may be coupled to the component-facing surface 22 of the rotary plate 25, and a translation coupler 40 formed in one or more bone anchoring components that are described or contemplated herein (e.g., the stem adapter 300 in the embodiment shown in FIGS. 10-15D).

The translation member 30 may also be referred to herein as a prosthesis translation member, a primary translation member, or a revision translation member.

The translation coupler 40 may also be referred to herein as a prosthesis translation coupler, a primary translation coupler, or a revision translation coupler.

In some embodiments, the translation member 30 may comprise a translation member body 35 that may project away from the component-facing surface 22 of the rotary plate 25 and a fastener hole 37 that may be formed therethrough.

In some embodiments, the translation member body 35 may comprise an elongate shape that may or may not span across the entire diameter of the rotary plate 25.

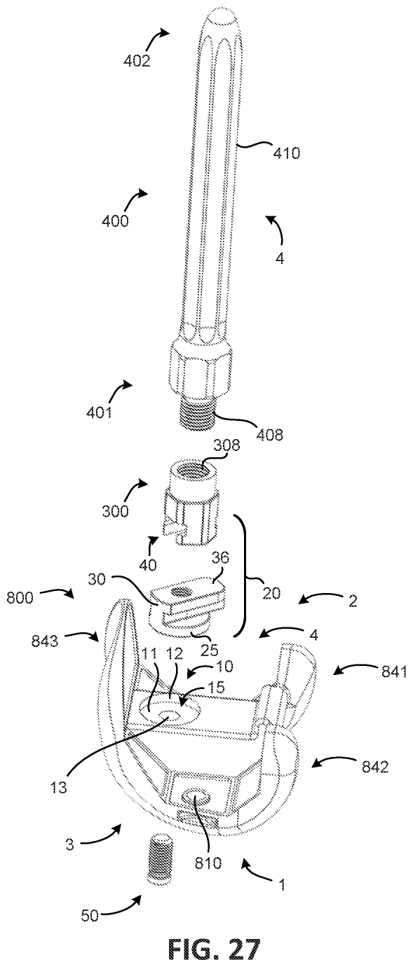

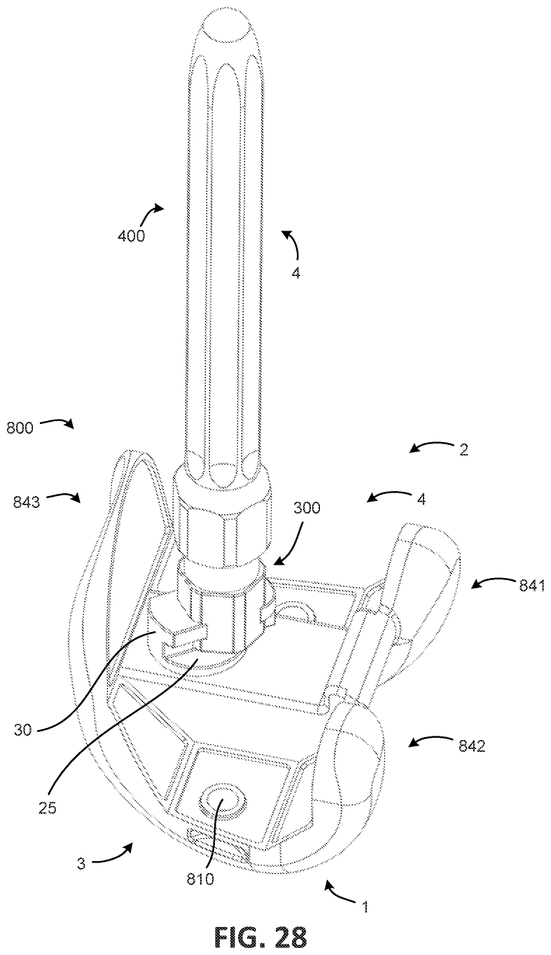

In some embodiments, the translation member body 35 may comprise an elongate shape that may span across more than the plate diameter 24 of the rotary plate 25 (e.g., see FIGS. 27 and 28 where the translation member 30 includes a projecting portion 36 spanning further than the plate diameter 24 of the rotary plate 25, on at least one side of the rotary plate 25).

Continuing with FIGS. 10-15D, the translation member 30 may also include a first translation member arm 31 projecting from a distal portion of the translation member body 35 along a first direction that may run alongside and/or generally parallel to the rotary plate 25. The translation member 30 may also include a second translation member arm 32 projecting from the distal portion of the translation member body 35 along a second direction, opposite the first direction, that may run alongside and/or generally parallel to the rotary plate 25.

In other embodiments, the translation member 30 may comprise an elongate coupling slot (not shown) that may be formed in the rotary plate 25 instead of the translation member body 35 that projects from the rotary plate 25. In these embodiments, the elongate coupling slot may be configured to translatably couple with a bone anchoring component to translatably couple the bone anchoring component to a bone-facing side of an arthroplasty prosthesis.

In some embodiments, the stem 400 may include the translation coupler 40 formed in a proximal end of the stem 400 (not shown).

In some embodiments, the stem adapter 300 may include the translation coupler 40 formed in a proximal end of the stem adapter 300, a bone engagement surface 4 that may or may not include a porous surface coating, a stem adapter bore 344, and a stem adapter thread 308.

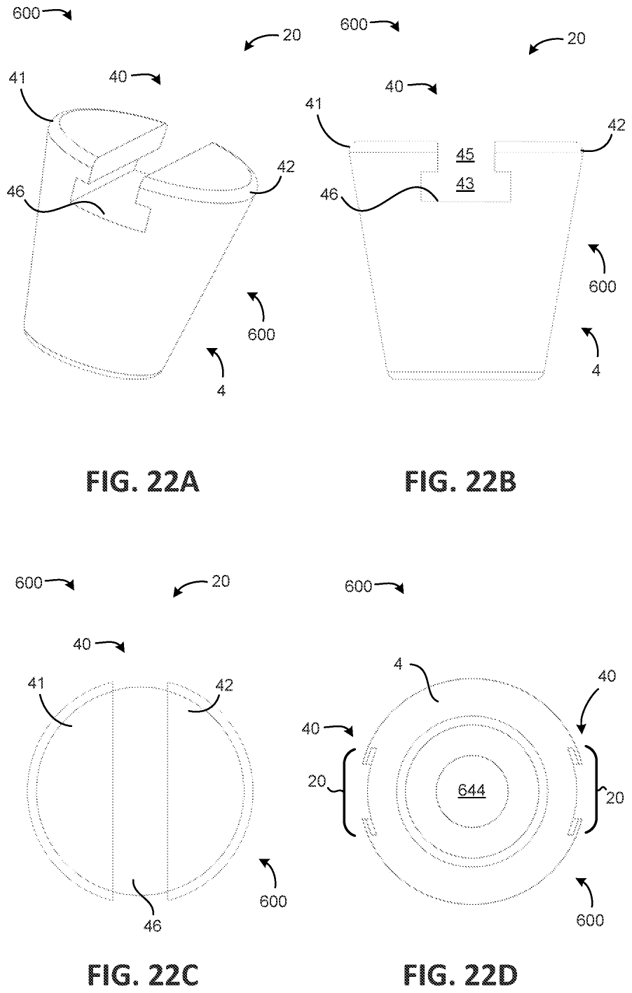

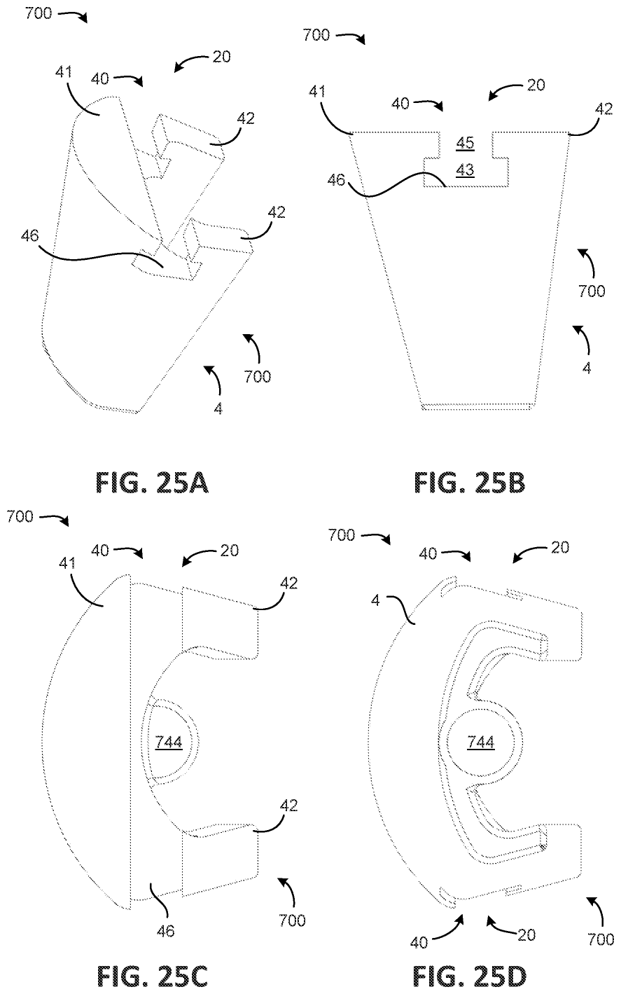

The translation coupler 40 morphology may be formed in the stem adapter 300 and/or formed in other components of the present disclosure, as will be discussed in more detail below with reference to FIGS. 22A-22D and 25A-25D.

Continuing with FIGS. 15A-15D, the translation coupler 40 formed in the stem adapter 300 may generally include a first translation coupler arm 41, a second translation coupler arm 42, a translation coupler arm slot 43, a translation coupler body slot 45, and a translation coupler lock surface 46.

The translation coupler arm slot 43 may be configured to receive the first translation member arm 31 and the second translation member arm 32 of the translation member 30 therein, and the translation coupler body slot 45 may be configured to receive the proximal/thinner portion of the translation member body 35 therein. In this manner, the stem adapter 300 may be configured to slidably couple and/or translatably mate with the translation member 30, as shown in FIG. 11.

In some embodiments, the rotary plate 25 may include the translation member 30 coupled to the rotary plate 25, and the bone anchoring component (e.g., the stem adapter 300 and/or the stem 400 in this embodiment) may comprise the translation coupler 40 configured to movably couple with the translation member 30 to enable the bone anchoring component to translate along the translation member 30 in a first direction with respect to the rotary plate 25, and translate along the translation member 30 in a second direction, opposite the first direction, with respect to the rotary plate 25. In these embodiments, the first adjustable attachment feature or prosthesis attachment feature 10 may also comprise the rotary recess 15 formed in the bone-facing side of the arthroplasty prosthesis, and the second adjustable attachment feature or component attachment feature 20 may comprise the rotary plate 25 which may be coupled to the bone anchoring component (e.g., the stem adapter 300 and/or the stem 400, via the translation member 30) and configured to mate with the rotary recess 15 to also rotatably couple the bone anchoring component to the bone-facing side of the arthroplasty prosthesis.

In this manner, the bone anchoring component (e.g., the stem adapter 300 and/or the stem 400) may both rotatably and/or translatably mate with the bone-facing side 2 of the tibial base plate 100. This arrangement allows the stem adapter 300 and/or the stem 400 to be positionally adjusted with respect to the bone-facing side 2 of the tibial base plate 100 along an infinite number of different rotational and/or translational positions (e.g., a rotational position along 360 degrees or rotation and/or a translational position along the length of the translation member 30) in order to achieve an exact desired position of the bone anchoring component with respect to the bone-facing side of the arthroplasty prosthesis.

Once a desired position of the bone anchoring component with respect to the bone-facing side of the arthroplasty prosthesis has been achieved, this desired position may be locked into place by tightening the locking fastener 50, as shown in FIGS. 12 and 13. For example, as the locking fastener 50 is tightened, the distal end of the locking fastener 50 may press against the translation coupler lock surface 46 and push the first and second translation coupler arms of the translation coupler 40 down against the first and second translation member arms of the translation member 30. This will frictionally lock the stem adapter 300 against the translation member 30 and prevent the stem adapter 300 from translating along the translation member 30. Moreover, tightening the locking fastener 50 will also tighten the rotary plate 25 against the rotary recess 15 formed in the tibial base plate 100 to prevent the stem adapter 300 (and/or the stem 400) from rotating with respect to the bone-facing side 2 of the tibial base plate 100.

In some embodiments, the locking fastener 50 may be loosened to allow the stem 400 to rotatably and/or translatably move with respect to the bone-facing side 2 of the tibial base plate 100. In this state, the stem 400 may then be inserted into a prepared bone tunnel or resected bone to engage a bone engagement surface 4 of the stem 400 with a resected surface of the bone (e.g., a surface of a bone tunnel formed in the bone to receive the stem 400, which may be a little smaller in diameter than a diameter of the stem 400 to assure a good press-fit). Pushing the stem 400 further into the bone tunnel may result in the transmission of at least one force between the resected surface of the bone and the bone engagement surface 4 of the stem 400 that may cause the stem 400 to movably self-align or self-locate with respect to the bone-facing side 2 of the tibial base plate 100, as the stem is pushed into the bone tunnel. The operator may also grasp the tibial base plate 100 and manually move it with respect to the stem 400 that is within the bone tunnel to further adjust a position of the tibial base plate 100 with respect to the bone-facing side 2 of the tibial base plate 100 (if desired). Once a desired position of the bone anchoring component with respect to the bone-facing side of the arthroplasty prosthesis has been achieved, this desired position may be locked into place by tightening the locking fastener 50, as previously discussed. In this manner, the self-adjusting and/or adjustable arthroplasty systems of the present disclosure may eliminate the need for trial instruments and/or eliminate the need to perform one or more trial procedures in order to select a prosthesis with a “closest fit” or a “closest discrete shape” from among a number of different prostheses in a kit having many different discrete sizes and shapes (e.g., each discrete prosthesis may include a different discrete rotational position, shape, or morphology, and/or a different discrete offset position, shape, or morphology).

In other embodiments, the structures of the second adjustable attachment feature or component attachment feature 20 and the first adjustable attachment feature or prosthesis attachment feature 10 may be reversed, such that the first adjustable attachment feature comprises the translation coupler 40 on the bone-facing side of the arthroplasty prosthesis, and the second adjustable attachment feature comprises the translation member 30 configured to movably couple with the translation coupler 40 and enable the bone anchoring component to translate along the translation coupler 40 in a first direction with respect to the bone-facing side of the arthroplasty prosthesis, and translate along the translation coupler in a second direction, opposite the first direction, with respect to the bone-facing side of the arthroplasty prosthesis.

In some embodiments, a knee arthroplasty system for replacing a natural articular surface on a bone of a knee joint may include a knee arthroplasty prosthesis, a primary bone anchoring component, and a revision bone anchoring component. The knee arthroplasty prosthesis may include a joint-facing side having an articular surface and a bone-facing side having a prosthesis attachment feature. The primary bone anchoring component may include a primary bone engagement surface securable to a resected surface of the bone and a primary attachment feature. At least a majority of the primary bone anchoring component may be located outside an intramedullary canal of the bone when the primary bone anchoring component is anchored to the bone. The revision bone anchoring component may include a revision attachment feature and a stem having a distal portion that may be securable within the intramedullary canal of the bone when the revision bone anchoring component is anchored to the bone. The prosthesis attachment feature may be configured to removably couple the primary bone anchoring component to the bone-facing side of the knee arthroplasty prosthesis by mating with the primary attachment feature. The prosthesis attachment feature may be configured to removably couple the revision bone anchoring component to the bone-facing side of the knee arthroplasty prosthesis by mating with the revision attachment feature.

In some embodiments, a knee arthroplasty system for replacing a natural articular surface on a bone of a knee joint may include a knee arthroplasty prosthesis, a primary bone anchoring component, and a revision bone anchoring component. The primary bone anchoring component may include a joint-facing side having an articular surface and a bone-facing side having a prosthesis attachment feature formed thereon. The primary bone anchoring component may include a primary bone engagement surface securable to a resected primary surface of the bone and a primary attachment feature. The revision bone anchoring component may include a revision bone engagement surface securable to a resected revision surface of the bone and a revision attachment feature. The prosthesis attachment feature may be configured to mate with either of the primary attachment feature and the revision attachment feature to couple either of the primary bone anchoring component and the revision bone anchoring component to the bone-facing side of the knee arthroplasty prosthesis. The prosthesis attachment feature may be configured to rotatably and/or translatably mate with at least one of the primary attachment feature and the revision attachment feature.

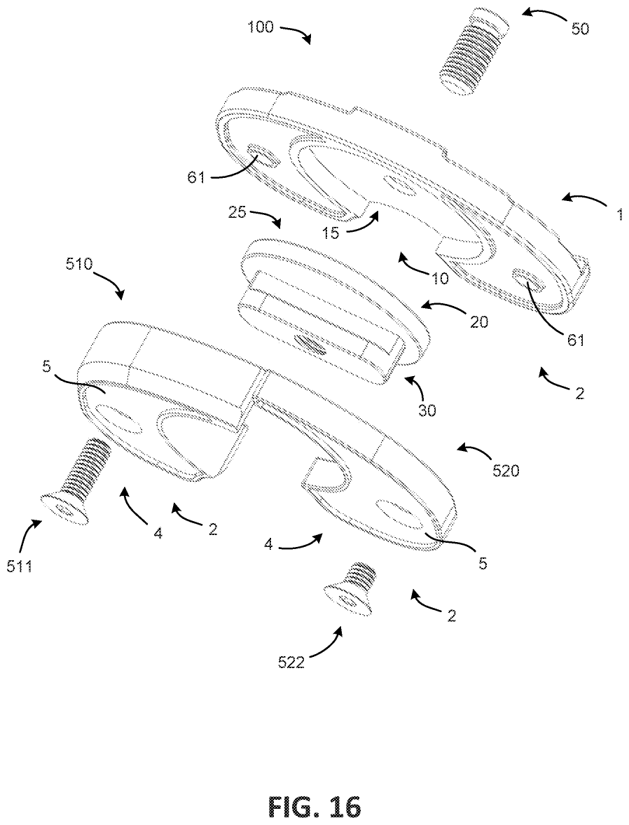

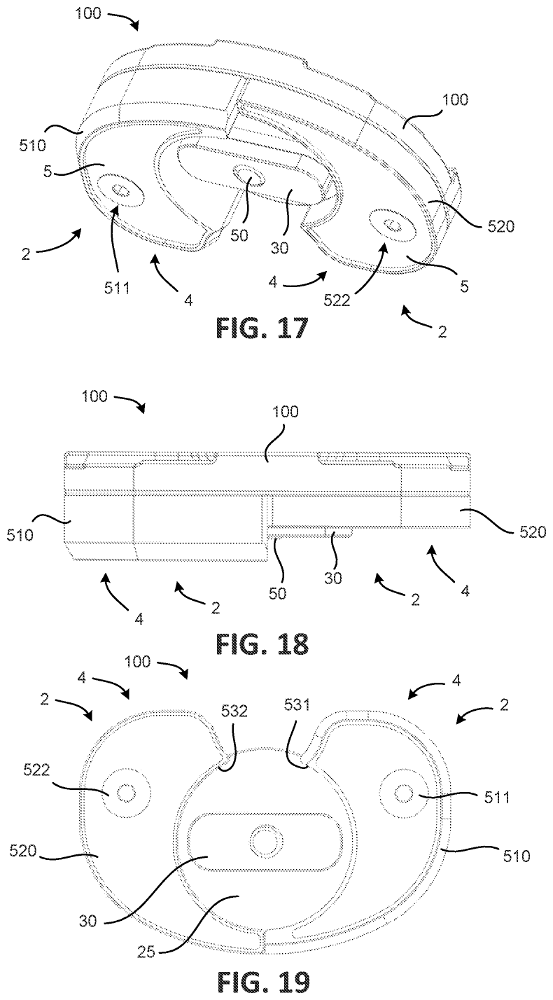

FIGS. 16-19 illustrate various views of an arthroplasty system or knee arthroplasty system, according to another embodiment of the present disclosure. Specifically, FIG. 16 is an exploded view of the arthroplasty system; FIG. 17 is a bottom perspective view of the arthroplasty system, after assembly; FIG. 18 is a front view of the arthroplasty system; and FIG. 19 is a bottom view of the arthroplasty system.

In some embodiments, the arthroplasty system of FIGS. 16-19 may generally include the locking fastener 50, the tibial base plate 100, at least a portion of the component attachment feature 20, a first augment 510 having a first augment ledge 531, a second augment 520 having a second augment ledge 532, a first augment fastener 511, and a second augment fastener 522.

In some embodiments, the locking fastener 50, the tibial base plate 100, and the component attachment feature 20 may comprise the same/similar features to other locking fasteners, tibial base plates, and component attachment features discussed herein.

In some embodiments, the arthroplasty system of FIGS. 16-19 may also include other components discussed herein, such as the stem 400, the stem adapter 300, etc.

In some embodiments, a bone-facing side 2 of the first augment 510 and/or the second augment 520 may include a bone engagement surface 4 comprising a porous surface coating 5. In some embodiments, the porous surface coating 5 may be an additive surface coating (e.g., a porous titanium surface, or other porous metal surface), a nano-coating, or other treatment that may be configured to promote bone ingrowth for cementless (or cemented) applications.

In some embodiments, a height of the first and/or second augments may be selected for a given resected tibial plateau morphology.

In the examples shown in FIGS. 16-19, a height of the first augment 510 may be about 10 mm, and a height of the second augment 520 may be about 5 mm. However, it will be understood that augments of any size/height may be utilized with the arthroplasty systems and components of the present disclosure.

As shown in FIG. 19, in some embodiments the first augment ledge 531 and/or the second augment ledge 532 may somewhat overhang the rotary plate 25 and help lock/retain the rotary plate 25 to the tibial base plate 100.

FIGS. 20-22D illustrate various views of an arthroplasty system or knee arthroplasty system, according to another embodiment of the present disclosure. Specifically, FIG. 20 is an exploded view of the arthroplasty system; FIG. 21 is a perspective view of the arthroplasty system; FIG. 22A is a perspective view of a cone-shaped sleeve of the arthroplasty system; FIG. 22B is a side view of the cone-shaped sleeve; FIG. 22C is a top view of the cone-shaped sleeve; and FIG. 22D is a bottom view of the cone-shaped sleeve.

In some embodiments, the arthroplasty system of FIGS. 20-22D may generally include the locking fastener 50, the tibial base plate 100, the component attachment feature 20, the stem 400, and a cone-shaped sleeve 600 or stem/cone augment.

In some embodiments, the locking fastener 50, the tibial base plate 100, the component attachment feature 20, and the stem 400 may comprise the same/similar features to other locking fasteners, tibial base plates, component attachment features, and stems discussed herein.

In some embodiments, the cone-shaped sleeve 600 may include the translation coupler 40 formed in a proximal end of the cone-shaped sleeve 600, a bone engagement surface 4 that may or may not include a porous surface coating 5, a cone-shaped sleeve bore 644, and a cone-shaped sleeve thread 608.

In some embodiments, the porous surface coating 5 may be an additive surface coating (e.g., a porous titanium surface, or other porous metal surface), a nano-coating, or other treatment that may be configured to promote bone ingrowth for cementless (or cemented) applications.

The translation coupler 40 formed in the proximal end of the cone-shaped sleeve 600 may perform the same function as the translation coupler 40 formed in the proximal end of the stem adapter 300, as previously discussed. In this manner, the cone-shaped sleeve 600 (and/or the stem 400 coupled thereto) may rotatably and/or translatably move with respect to the bone-facing side 2 of the tibial base plate 100, similar to that previously described with respect to FIGS. 10-15D.

In some embodiments, the arthroplasty system of FIGS. 20-22D may be utilized for revision knee arthroplasty procedures that may require the stem 400 (or an intramedullary rod), and the cone-shaped sleeve 600 for extra stability. However, it will also be understood that the arthroplasty system of FIGS. 20-22D may also be utilized for primary knee arthroplasty procedures as well.

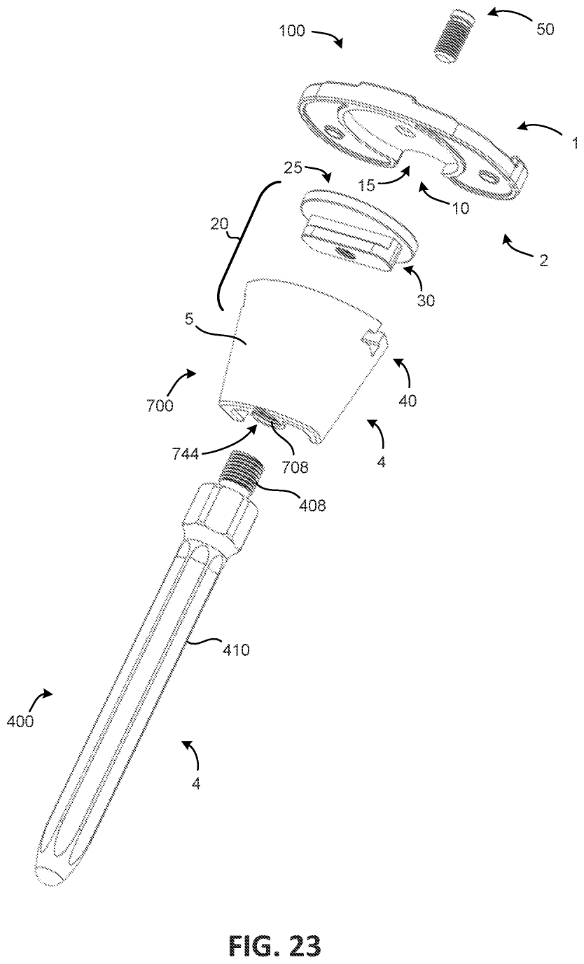

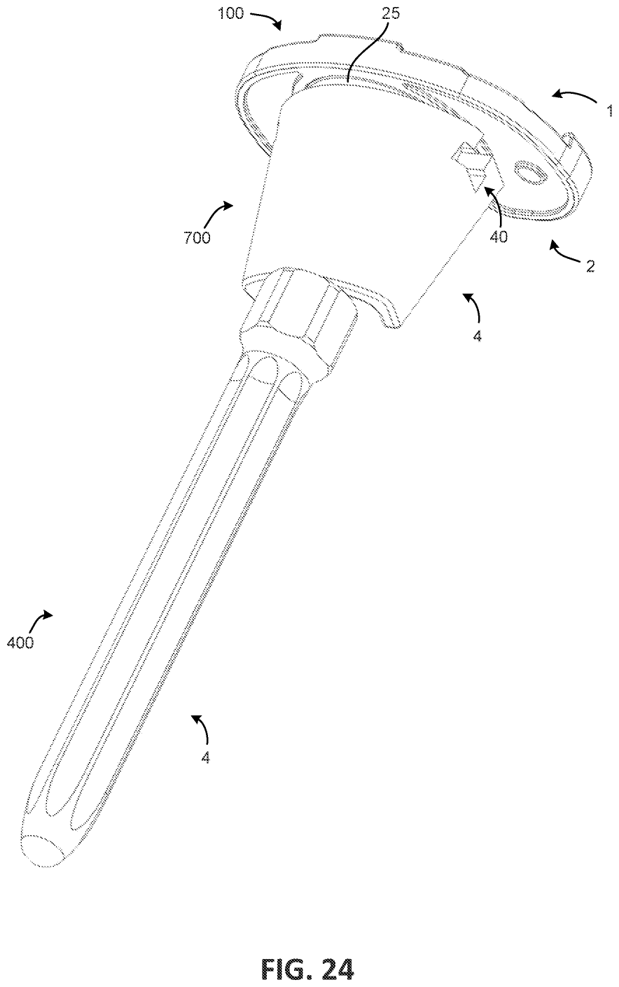

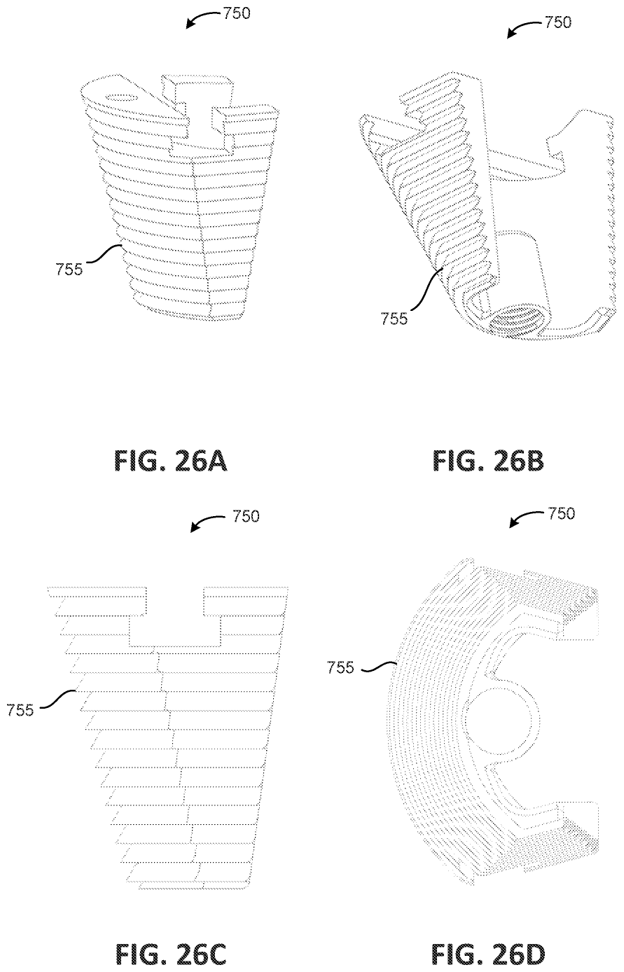

FIGS. 23-26D illustrate various views of an arthroplasty system or knee arthroplasty system, according to another embodiment of the present disclosure. Specifically, FIG. 23 illustrates an exploded view of the arthroplasty system; FIG. 24 is a perspective view of the arthroplasty system, after assembly; FIG. 25A is a perspective view of an oblong sleeve of the arthroplasty system; FIG. 25B is a side view of the oblong sleeve; FIG. 25C is a top view of the oblong sleeve; FIG. 25D is a bottom view of the oblong sleeve; FIG. 26A is a perspective view of an example broach of the arthroplasty system; FIG. 26B is another perspective view of the broach; FIG. 26C is a side view of the broach; and FIG. 26D is a bottom view of the broach.

In some embodiments, the arthroplasty system of FIGS. 23-26D may generally include the locking fastener 50, the tibial base plate 100, the component attachment feature 20, the stem 400, and an oblong sleeve 700.

In some embodiments, the locking fastener 50, the tibial base plate 100, the component attachment feature 20, and the stem 400 may comprise the same/similar features to other locking fasteners, tibial base plates, component attachment features, and stems discussed herein.

In some embodiments, the oblong sleeve 700 may include the translation coupler 40 formed in a proximal end of the oblong sleeve 700, a bone engagement surface 4 that may or may not include a porous surface coating 5, an oblong sleeve bore 744, and an oblong sleeve thread 708.

In some embodiments, the porous surface coating 5 may be an additive surface coating (e.g., a porous titanium surface, or other porous metal surface), a nano-coating, or other treatment that may be configured to promote bone ingrowth for cementless (or cemented) applications.

The translation coupler 40 formed in the proximal end of the oblong sleeve 700 may perform the same function as the translation coupler 40 formed in the proximal end of the stem adapter 300, as previously discussed. In this manner, the oblong sleeve 700 (and/or the stem 400 coupled thereto) may rotatably and/or translatably move with respect to the bone-facing side 2 of the tibial base plate 100, similar to that previously described with respect to FIGS. 10-15D.

In some embodiments, the arthroplasty system of FIGS. 23-26D may be utilized for revision knee arthroplasty procedures that may require the stem 400 (or an intramedullary rod), and the oblong sleeve 700 for extra stability. However, it will also be understood that the arthroplasty system of FIGS. 23-26D may also be utilized for primary knee arthroplasty procedures as well.

FIGS. 26A-26D show various views of a broach 750 that may be utilized in conjunction with the oblong sleeve 700. The broach 750 may include the same general shape as the oblong sleeve 700 shown in FIGS. 25A-25D. However, the broach 750 may also include one or more cutting edges 755 disposed about the body of the broach 750 and configured to cut/rasp a surface of a bone (e.g., a tibia, a femur, etc.) to prepare the bone to receive the oblong sleeve 700.

In some embodiments, the broach 750 may be somewhat smaller in size than a corresponding oblong sleeve of the arthroplasty system to ensure a tight fit when the oblong sleeve 700 is press-fit in a bone that has been prepared by the broach 750.

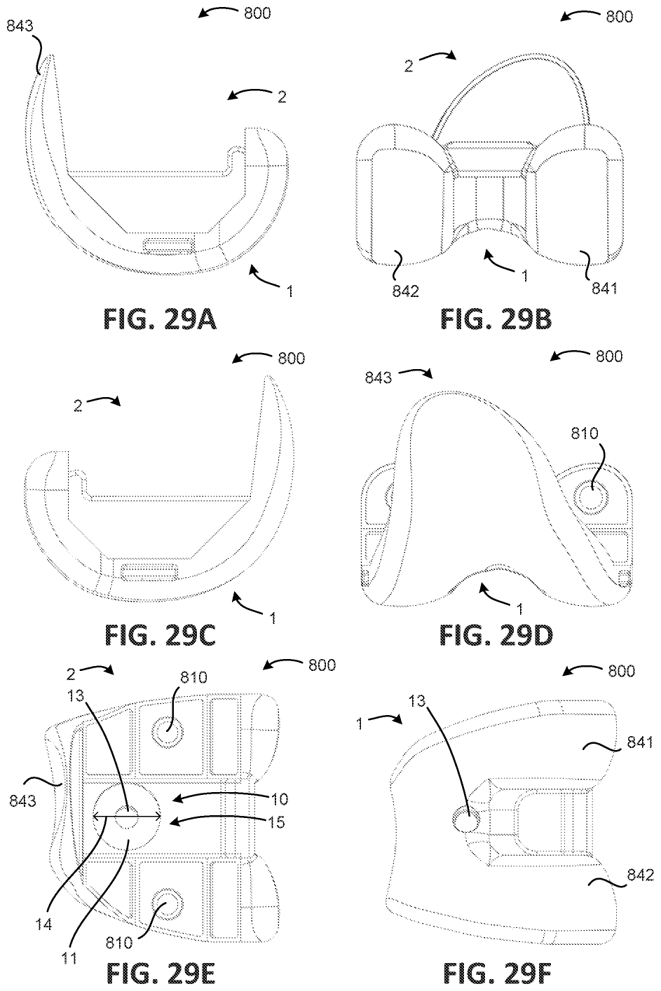

FIGS. 27-29F illustrate various views of an arthroplasty system or knee arthroplasty system, according to another embodiment of the present disclosure. Specifically, FIG. 27 is an exploded view of the arthroplasty system; FIG. 28 is a perspective view of the arthroplasty system, after assembly; FIG. 29A is a lateral side view of a femoral prosthesis of the arthroplasty system; FIG. 29B is a rear view of the femoral prosthesis; FIG. 29C is a medial side view of the femoral prosthesis; FIG. 29D is a front view of the femoral prosthesis; FIG. 29E is a top view of the femoral prosthesis; and FIG. 29F is a bottom view of the femoral prosthesis.

In some embodiments, the arthroplasty system of FIGS. 27-29F may generally include the locking fastener 50, the component attachment feature 20, the stem adapter 300, the stem 400, and a femoral component or femoral prosthesis 800.

In some embodiments, the locking fastener 50, the component attachment feature 20, the stem adapter 300, and the stem 400 may comprise the same/similar features to other locking fasteners, component attachment features, stem adapters, and stems discussed herein.

In some embodiments, the femoral prosthesis 800 may generally include a joint-facing side 1 comprising an articular surface 3, a femoral articular surface, a first condyle articular surface 841, a second condyle articular surface 842, and/or a patellar articulating surface 843. The femoral prosthesis 800 may also include a bone-facing side 2 comprising a bone engagement surface 4 that may or may not include a porous surface coating for cementless applications, and one or more augment connection interfaces 810.

In some embodiments, the porous surface coating may be an additive surface coating (e.g., a porous titanium surface, or other porous metal surface), a nano-coating, or other treatment that may be configured to promote bone ingrowth for cementless (or cemented) applications.

In some embodiments, the arthroplasty system of FIGS. 27-29F may be utilized for revision knee arthroplasty procedures that may require the stem 400 or an intramedullary rod, for extra stability. However, it will also be understood that the arthroplasty system of FIGS. 27-29F may also be utilized for primary knee arthroplasty procedures as well.

In some embodiments, the femoral prosthesis 800 may also include the prosthesis attachment feature 10 or first adjustable attachment feature, as previously discussed herein with respect to FIGS. 1-5D. The prosthesis attachment feature 10 may be formed in/on the bone-facing side 2 of the femoral prosthesis 800 and a size of the recess diameter 14 may be adapted to fit the femoral prosthesis 800.

In some embodiments, the prosthesis attachment feature 10 may comprise a circular shape having a diameter of at least 10 mm.

However, in some embodiments the prosthesis attachment feature 10 may also comprise a circular shape having a diameter that is less than 10 mm.

In some embodiments, the prosthesis attachment feature 10 may comprise a circular shape having a diameter of at least 20 mm.

In some embodiments, the prosthesis attachment feature 10 may comprise a circular shape having a diameter of at least 30 mm.

In some embodiments, the prosthesis attachment feature 10 may comprise a circular shape having a diameter of at least 40 mm.

In some embodiments, the prosthesis attachment feature 10 may comprise a circular shape having a diameter of at least 50 mm.

The size of the component attachment feature 20 may also be adapted to fit the recess diameter 14. This may result in the translation member 30 including the projecting portion 36, as previously discussed.

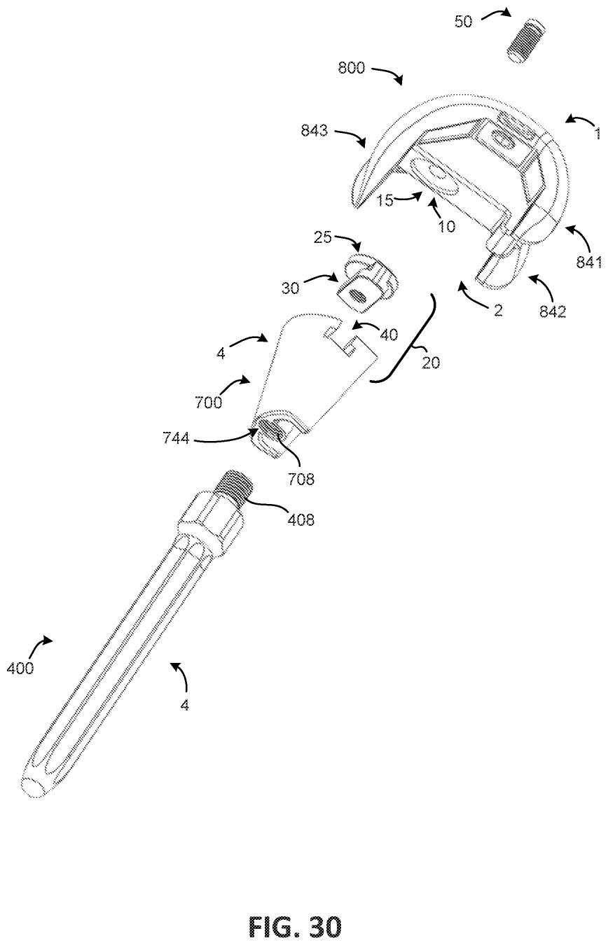

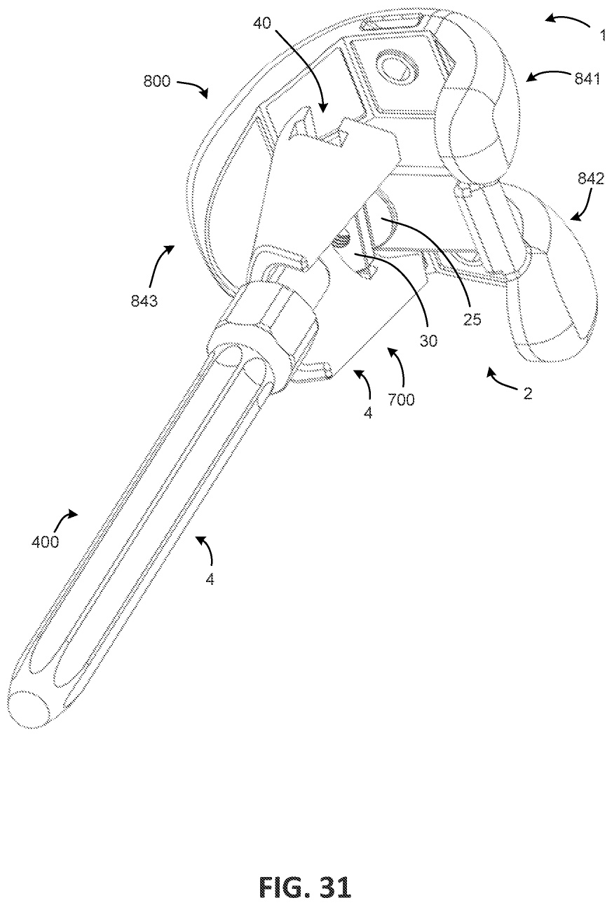

FIGS. 30 and 31 illustrate an exploded view and an assembled perspective view of an arthroplasty system or knee arthroplasty system, according to another embodiment of the present disclosure.

In some embodiments, the arthroplasty system of FIGS. 30 and 31 may generally include the locking fastener 50, the femoral prosthesis 800, the component attachment feature 20, the oblong sleeve 700, and the stem 400, as previously described herein.

In some embodiments, the arthroplasty system of FIGS. 30 and 31 may be utilized for revision knee arthroplasty procedures that may require the stem 400, an intramedullary rod, and/or the oblong sleeve 700 for extra stability. However, it will also be understood that the arthroplasty system of FIGS. 30 and 31 may also be utilized for primary knee arthroplasty procedures as well.

For example, in some embodiments the arthroplasty system of FIGS. 30 and 31 may be modified to only include the locking fastener 50, the femoral prosthesis 800, the component attachment feature 20, and the oblong sleeve 700 (or the cone-shaped sleeve 600, or a shorter stem, or another sleeve or keel, etc.), but not the stem 400 that is shown in FIGS. 30 and 31. In these embodiments, the arthroplasty system may be a referred to as a primary arthroplasty system that may be utilized for a primary arthroplasty procedure to repair a diseased knee joint that may or may not have experienced a previous arthroplasty procedure. In any case, the femoral bone may still be healthy enough to support a primary arthroplasty system that does not need the stem 400 (or another long stem or femoral intramedullary rod, etc.) to provide the extra stability that a revision arthroplasty procedure may require.

In some embodiments, a method for replacing a natural articular surface on a bone may include: movably mating a first adjustable attachment feature of an arthroplasty prosthesis with a second adjustable attachment feature of a bone anchoring component to movably couple the bone anchoring component to a bone-facing side of the arthroplasty prosthesis; securing a bone engagement surface of the bone anchoring component to a resected surface of the bone; and replacing the natural articular surface on the bone with an articular surface on a joint-facing side of the of the arthroplasty prosthesis.

In some embodiments, movably mating the first adjustable attachment feature with the second adjustable attachment feature may include inserting a rotary plate of the first adjustable attachment feature into a rotary recess of the second adjustable attachment feature to rotatably couple the bone anchoring component to the bone-facing side of the arthroplasty prosthesis.

In some embodiments, movably mating the first adjustable attachment feature with the second adjustable attachment feature may include inserting a rotary plate of the second adjustable attachment feature into a rotary recess of the first adjustable attachment feature to rotatably couple the bone anchoring component to the bone-facing side of the arthroplasty prosthesis.

In some embodiments, movably mating the first adjustable attachment feature with the second adjustable attachment feature may include mating a translation coupler of the first adjustable attachment feature with a translation member of the second adjustable attachment feature to translatably couple the bone anchoring component to the bone-facing side of the arthroplasty prosthesis.

In some embodiments, movably mating the first adjustable attachment feature with the second adjustable attachment feature may include mating a translation coupler of the second adjustable attachment feature with a translation member of the first adjustable attachment feature to translatably couple the bone anchoring component to the bone-facing side of the arthroplasty prosthesis.

In some embodiments, movably mating the first adjustable attachment feature with the second adjustable attachment feature may include inserting a rotary plate of the second adjustable attachment feature into a rotary recess of the first adjustable attachment feature to rotatably couple the bone anchoring component to the bone-facing side of the arthroplasty prosthesis, and mating a translation coupler of the bone anchoring component with a translation member of the rotary plate to translatably couple the bone anchoring component to the bone-facing side of the arthroplasty prosthesis.

In some embodiments, the method may also include engaging the bone engagement surface of the bone anchoring component with the resected surface of the bone and transmitting at least one force between the resected surface and the bone engagement surface to movably self-locate the bone anchoring component with respect to the bone-facing side of the arthroplasty prosthesis.

In some embodiments, the method may also include activating a locking mechanism, such as the locking fastener 50 as one non-limiting example, to prevent further movement of the bone anchoring component with respect to the bone-facing side of the arthroplasty prosthesis.

In some embodiments, a method for replacing a natural articular surface on a bone of a knee joint may include: mating a prosthesis attachment feature of a knee arthroplasty prosthesis with one of a primary attachment feature of a primary bone anchoring component and a revision attachment feature of a revision bone anchoring component to removably couple one of the primary bone anchoring component and the revision bone anchoring component to a bone-facing side of the knee arthroplasty prosthesis. The method may also include securing one of a primary bone engagement surface of the primary bone anchoring component and a revision bone engagement surface of the revision bone anchoring component to a resected surface of the bone and replacing the natural articular surface on the bone with an articular surface on a joint-facing side of the of the knee arthroplasty prosthesis. At least a majority of the primary bone anchoring component may be located outside an intramedullary canal of the bone when the primary bone anchoring component is secured to the bone. A distal portion of a stem of the revision bone anchoring component may be securable within the intramedullary canal of the bone when the revision bone anchoring component is secured to the bone.

In some embodiments, mating the prosthesis attachment feature with one of the primary attachment feature and the revision attachment feature may include inserting a prosthesis plate of the prosthesis attachment feature into one of a primary recess of the primary attachment feature and a revision recess of the revision attachment feature to removably couple one of the primary bone anchoring component and the revision bone anchoring component with the bone-facing side of the knee arthroplasty prosthesis.

In some embodiments, mating the prosthesis attachment feature with one of the primary attachment feature and the revision attachment feature may include mating a prosthesis recess of the prosthesis attachment feature with one of a primary plate of the primary attachment feature and a revision plate of the revision attachment feature to removably couple one of the primary bone anchoring component and the revision bone anchoring component with the bone-facing side of the knee arthroplasty prosthesis.

In some embodiments, mating the prosthesis attachment feature with one of the primary attachment feature and the revision attachment feature may include mating a prosthesis translation member of the prosthesis attachment feature with one of a primary translation coupler of the primary attachment feature and a revision translation coupler of the revision attachment feature to removably couple one of the primary bone anchoring component and the revision bone anchoring component with the bone-facing side of the knee arthroplasty prosthesis.

In some embodiments, mating the prosthesis attachment feature with one of the primary attachment feature and the revision attachment feature may include mating a prosthesis translation coupler of the prosthesis attachment feature with one of a primary translation member of the primary attachment feature and a revision translation member of the revision attachment feature to removably couple one of the primary bone anchoring component and the revision bone anchoring component with the bone-facing side of the knee arthroplasty prosthesis.

In some embodiments, the method may also include engaging one of the primary bone engagement surface and the revision bone engagement surface with the resected surface of the bone and transmitting at least one force between the resected surface and one of the primary bone engagement surface and the revision bone engagement surface to movably self-locate one of the primary bone anchoring component and the revision bone anchoring component with respect to the bone-facing side of the knee arthroplasty prosthesis.

In some embodiments, the method may also include activating a locking mechanism, such as the locking fastener 50 as one non-limiting example, to prevent further movement of one of the primary bone anchoring component and the revision bone anchoring component with respect to the bone-facing side of the knee arthroplasty prosthesis.

In some embodiments, the knee arthroplasty prosthesis may include one of: a tibial base plate having a tibial articular surface on the joint-facing side of the tibial base plate; and a femoral prosthesis having a femoral articular surface on the joint-facing side of the femoral prosthesis. The primary bone anchoring component may include at least one of: a short stem, a keel, a sleeve, an adapter, and an augment.

In some embodiments, the revision bone anchoring component may also include at least one of: a keel, a sleeve, an adapter, and an augment.

In some embodiments, a method for replacing a natural articular surface on a bone of a knee joint may include selecting either of: a primary bone anchoring component having a primary bone engagement surface securable to a resected surface of the bone and a primary attachment feature; and a revision bone anchoring component having a revision bone engagement surface securable to the resected surface of the bone and a revision attachment feature. The method may also include rotatably and/or translatably mating a prosthesis attachment feature of a knee arthroplasty prosthesis with either of the primary attachment feature and the revision attachment feature to couple either of the primary bone anchoring component and the revision bone anchoring component to a bone-facing side of the knee arthroplasty prosthesis. The method may also include securing either of the primary bone engagement surface and the revision bone engagement surface to the resected surface of the bone and replacing the natural articular surface on the bone with an articular surface on a joint-facing side of the of the knee arthroplasty prosthesis.

In some embodiments, rotatably and/or translatably mating the prosthesis attachment feature with either of the primary attachment feature and the revision attachment feature may include inserting a prosthesis plate of the prosthesis attachment feature into either of a primary recess of the primary attachment feature and a revision recess of the revision attachment feature to couple either of the primary bone anchoring component and the revision bone anchoring component to the bone-facing side of the knee arthroplasty prosthesis.

In some embodiments, rotatably and/or translatably mating the prosthesis attachment feature with either of the primary attachment feature and the revision attachment feature may include mating a prosthesis recess of the prosthesis attachment feature with either of a primary plate of the primary attachment feature and a revision plate of the revision attachment feature to couple either of the primary bone anchoring component and the revision bone anchoring component to the bone-facing side of the knee arthroplasty prosthesis.

In some embodiments, rotatably and/or translatably mating the prosthesis attachment feature with either of the primary attachment feature and the revision attachment feature may include mating a prosthesis translation member of the prosthesis attachment feature with either of a primary translation coupler of the primary attachment feature and a revision translation coupler of the revision attachment feature to removably couple either of the primary bone anchoring component and the revision bone anchoring component with the bone-facing side of the knee arthroplasty prosthesis.

In some embodiments, rotatably and/or translatably mating the prosthesis attachment feature with either of the primary attachment feature and the revision attachment feature may include mating a prosthesis translation coupler of the prosthesis attachment feature with either of a primary translation member of the primary attachment feature and a revision translation member of the revision attachment feature to removably couple either of the primary bone anchoring component and the revision bone anchoring component with the bone-facing side of the knee arthroplasty prosthesis.

In some embodiments, the method may also include engaging either of the primary bone engagement surface and the revision bone engagement surface with the resected surface of the bone and transmitting at least one force between the resected surface and either of the primary bone engagement surface and the revision bone engagement surface to movably self-locate either of the primary bone anchoring component and the revision bone anchoring component with respect to the bone-facing side of the knee arthroplasty prosthesis.

In some embodiments, the method may also include activating a locking mechanism, such as the locking fastener 50 as one non-limiting example, to prevent further movement of either of the primary bone anchoring component and the revision bone anchoring component with respect to the bone-facing side of the knee arthroplasty prosthesis.

In some embodiments, activating the locking mechanism may include rotating the locking fastener 50 in a first direction to prevent further movement of either of the primary bone anchoring component and the revision bone anchoring component with respect to the bone-facing side of the knee arthroplasty prosthesis.

In some embodiments, releasing the locking mechanism may include rotating the locking fastener 50 in a second direction to allow movement of either of the primary bone anchoring component and the revision bone anchoring component with respect to the bone-facing side of the knee arthroplasty prosthesis.

In some embodiments, at least a majority of the primary bone anchoring component may be located outside an intramedullary canal of the bone when the primary bone anchoring component is secured to the bone, and a distal portion of a stem of the revision bone anchoring component may be securable within the intramedullary canal of the bone when the revision bone anchoring component is secured to the bone.

In some embodiments, the revision bone anchoring component may also include at least one of: a keel, a sleeve, an adapter, and an augment.

In some embodiments, the primary bone anchoring component may include at least one of: a short stem, a keel, a sleeve, an adapter, and an augment.

In some embodiments, the knee arthroplasty prosthesis may include a tibial base plate having a tibial articular surface on the joint-facing side of the tibial base plate.

In some embodiments, the knee arthroplasty prosthesis may include a femoral prosthesis having a femoral articular surface on the joint-facing side of the femoral prosthesis.

In some embodiments, a method for replacing a natural articular surface on a bone may include removably mating a prosthesis attachment feature of an arthroplasty prosthesis with a component attachment feature of a bone anchoring component to removably couple the bone anchoring component to a bone-facing side of the arthroplasty prosthesis. The method may also include securing a bone engagement surface of the bone anchoring component to a resected surface of the bone and replacing the natural articular surface on the bone with an articular surface on a joint-facing side of the of the arthroplasty prosthesis. The prosthesis attachment feature may also include a circular shape having a diameter of at least 10 mm.

In some embodiments, the prosthesis attachment feature may include a first circular shape having a first diameter of at least 10 mm, the component attachment feature may include a second circular shape having a second diameter of at least 10 mm, and the prosthesis attachment feature and the component attachment feature may include complementary circular shapes with each other.

In some embodiments, the diameter of the circular shape may be less than 10 mm.

In some embodiments, the diameter of the circular shape may be at least 15 mm.

In some embodiments, the diameter of the circular shape may be at least 20 mm.

In some embodiments, the diameter of the circular shape may be at least 25 mm.

In some embodiments, the diameter of the circular shape may be at least 30 mm.

In some embodiments, the diameter of the circular shape may be at least 35 mm.

In some embodiments, the diameter of the circular shape may be at least 40 mm.

In some embodiments, the diameter of the circular shape may be at least 45 mm.

In some embodiments, the diameter of the circular shape may be at least 50 mm.

In some embodiments, the diameter of the circular shape may be at least 55 mm.

In some embodiments, the diameter of the circular shape may be at least 60 mm.

In some embodiments, the diameter of the circular shape may be at least 65 mm.

In some embodiments, the diameter of the circular shape may be at least 70 mm.

In some embodiments, the diameter of the circular shape may be at least 75 mm.

In some embodiments, the diameter of the circular shape may be at least 10% of the medial-lateral width or anterior-posterior width of the arthroplasty prosthesis comprising the circular shape.

In some embodiments, the diameter of the circular shape may be at least 15% of the medial-lateral width or anterior-posterior width of the arthroplasty prosthesis comprising the circular shape.

In some embodiments, the diameter of the circular shape may be at least 20% of the medial-lateral width or anterior-posterior width of the arthroplasty prosthesis comprising the circular shape.

In some embodiments, the diameter of the circular shape may be at least 25% of the medial-lateral width or anterior-posterior width of the arthroplasty prosthesis comprising the circular shape.

In some embodiments, the diameter of the circular shape may be at least 30% of the medial-lateral width or anterior-posterior width of the arthroplasty prosthesis comprising the circular shape.

In some embodiments, the diameter of the circular shape may be at least 35% of the medial-lateral width or anterior-posterior width of the arthroplasty prosthesis comprising the circular shape.

In some embodiments, the diameter of the circular shape may be at least 40% of the medial-lateral width or anterior-posterior width of the arthroplasty prosthesis comprising the circular shape.

In some embodiments, the diameter of the circular shape may be at least 45% of the medial-lateral width or anterior-posterior width of the arthroplasty prosthesis comprising the circular shape.

In some embodiments, the diameter of the circular shape may be at least 50% of the medial-lateral width or anterior-posterior width of the arthroplasty prosthesis comprising the circular shape.

In some embodiments, the diameter of the circular shape may be at least 55% of the medial-lateral width or anterior-posterior width of the arthroplasty prosthesis comprising the circular shape.

In some embodiments, the diameter of the circular shape may be at least 60% of the medial-lateral width or anterior-posterior width of the arthroplasty prosthesis comprising the circular shape.

In some embodiments, the diameter of the circular shape may be at least 65% of the medial-lateral width or anterior-posterior width of the arthroplasty prosthesis comprising the circular shape.

In some embodiments, the diameter of the circular shape may be at least 70% of the medial-lateral width or anterior-posterior width of the arthroplasty prosthesis comprising the circular shape.

In some embodiments, the diameter of the circular shape may be at least 75% of the medial-lateral width or anterior-posterior width of the arthroplasty prosthesis comprising the circular shape.

In some embodiments, removably mating the prosthesis attachment feature with the component attachment feature may include inserting a prosthesis plate of the prosthesis attachment feature into a component recess of the component attachment feature to removably couple the bone anchoring component to the bone-facing side of the arthroplasty prosthesis.

In some embodiments, the method may also include engaging the bone engagement surface with the resected surface of the bone and transmitting at least one force between the resected surface and the bone engagement surface to movably self-locate the bone anchoring component with respect to the bone-facing side of the arthroplasty prosthesis.

In some embodiments, the method may also include activating a locking mechanism, such as the locking fastener 50 as one non-limiting example, to prevent further movement of the bone anchoring component with respect to the bone-facing side of the arthroplasty prosthesis.

In some embodiments, activating the locking mechanism may include rotating a locking fastener 50 in a first direction to prevent further movement of the bone anchoring component with respect to the bone-facing side of the arthroplasty prosthesis.

In some embodiments, releasing the locking mechanism may include rotating the locking fastener 50 in a second direction to allow movement of either of the primary bone anchoring component and the revision bone anchoring component with respect to the bone-facing side of the knee arthroplasty prosthesis.

In some embodiments, the method may also include engaging at least one first anti-rotation feature of the arthroplasty prosthesis with at least one second anti-rotation feature of the bone anchoring component to resist rotation of the bone anchoring component with respect to the bone-facing side of the arthroplasty prosthesis.

In some embodiments, the arthroplasty prosthesis may include one of: a tibial base plate having a tibial articular surface on the joint-facing side of the tibial base plate; and a femoral prosthesis having a femoral articular surface on the joint-facing side of the femoral prosthesis. The bone anchoring component may also include at least one of: a stem, a keel, a sleeve, an adapter, and an augment.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, methods, and/or instruments disclosed herein.

What is claimed is:

1. An arthroplasty system for replacing a natural articular surface on a bone, the arthroplasty system comprising:
an arthroplasty prosthesis comprising:
a joint-facing side comprising an articular surface; and
a bone-facing side comprising a first adjustable attachment feature; and
a bone anchoring component comprising:
a bone engagement surface securable to a resected surface of the bone; and
a second adjustable attachment feature;
wherein the first adjustable attachment feature is configured to movably mate with the second adjustable attachment feature to movably couple the bone anchoring component to the bone-facing side of the arthroplasty prosthesis.

2. The arthroplasty system of claim 1, wherein:
the first adjustable attachment feature comprises a rotary plate coupled to the bone-facing side of the arthroplasty prosthesis; and
the second adjustable attachment feature comprises a rotary recess formed in the bone anchoring component and configured to receive the rotary plate therein to rotatably couple the bone anchoring component to the bone-facing side of the arthroplasty prosthesis.

3. The arthroplasty system of claim 1, wherein:
the first adjustable attachment feature comprises a rotary recess formed in the bone-facing side of the arthroplasty prosthesis; and
the second adjustable attachment feature comprises a rotary plate coupled to the bone anchoring component and configured to mate with the rotary recess to rotatably couple the bone anchoring component to the bone-facing side of the arthroplasty prosthesis.

4. The arthroplasty system of claim 1, wherein:
the first adjustable attachment feature comprises a translation member coupled to the bone-facing side of the arthroplasty prosthesis; and
the second adjustable attachment feature comprises a translation coupler configured to movably couple with the translation member and enable the bone anchoring component to:
translate along the translation member in a first direction with respect to the bone-facing side of the arthroplasty prosthesis; and
translate along the translation member in a second direction, opposite the first direction, with respect to the bone-facing side of the arthroplasty prosthesis.

5. The arthroplasty system of claim 1, wherein:
the first adjustable attachment feature comprises a translation coupler on the bone-facing side of the arthroplasty prosthesis; and
the second adjustable attachment feature comprises a translation member configured to movably couple with the translation coupler and enable the bone anchoring component to:
translate along the translation coupler in a first direction with respect to the bone-facing side of the arthroplasty prosthesis; and
translate along the translation coupler in a second direction, opposite the first direction, with respect to the bone-facing side of the arthroplasty prosthesis.

6. The arthroplasty system of claim 1, wherein:
the first adjustable attachment feature comprises a rotary recess formed in the bone-facing side of the arthroplasty prosthesis; and
the second adjustable attachment feature comprises a rotary plate coupled to the bone anchoring component and configured to mate with the rotary recess to rotatably couple the bone anchoring component to the bone-facing side of the arthroplasty prosthesis;
wherein:
the rotary plate further comprises a translation member coupled to the rotary plate; and
the bone anchoring component further comprises a translation coupler configured to movably couple with the translation member and enable the bone anchoring component to:
translate along the translation member in a first direction with respect to the rotary plate; and
translate along the translation member in a second direction, opposite the first direction, with respect to the rotary plate.

7. A knee arthroplasty system for replacing a natural articular surface on a bone of a knee joint, the knee arthroplasty system comprising:
a knee arthroplasty prosthesis comprising:
a joint-facing side comprising an articular surface; and
a bone-facing side comprising a prosthesis attachment feature;
a primary bone anchoring component comprising:
a primary bone engagement surface securable to a resected surface of the bone; and
a primary attachment feature;
wherein at least a majority of the primary bone anchoring component is located outside an intramedullary canal of the bone when the primary bone anchoring component is anchored to the bone; and
a revision bone anchoring component comprising:
a revision attachment feature; and
a stem comprising a distal portion securable within the intramedullary canal of the bone when the revision bone anchoring component is anchored to the bone; and
wherein:
the prosthesis attachment feature is configured to removably couple the primary bone anchoring component to the bone-facing side of the knee arthroplasty prosthesis by mating with the primary attachment feature; and
the prosthesis attachment feature is configured to removably couple the revision bone anchoring component to the bone-facing side of the knee arthroplasty prosthesis by mating with the revision attachment feature.

8. The knee arthroplasty system of claim 7, wherein:
the prosthesis attachment feature comprises a prosthesis plate coupled to the bone-facing side of the knee arthroplasty prosthesis;
the primary attachment feature comprises a primary recess formed in the primary bone anchoring component and configured to removably mate with the prosthesis plate; and
the revision attachment feature comprises a revision recess formed in the revision bone anchoring component and configured to removably mate with the prosthesis plate.

9. The knee arthroplasty system of claim 7, wherein:
the prosthesis attachment feature comprises a prosthesis recess formed in the bone-facing side of the knee arthroplasty prosthesis;
the primary attachment feature comprises a primary plate coupled to the primary bone anchoring component and configured to removably mate with the prosthesis recess; and
the revision attachment feature comprises a revision plate coupled to the revision bone anchoring component and configured to removably mate with the prosthesis recess.

10. The knee arthroplasty system of claim 7, wherein:
the prosthesis attachment feature comprises a prosthesis translation member on the bone-facing side of the knee arthroplasty prosthesis;
the primary attachment feature comprises a primary translation coupler configured to removably mate with the prosthesis translation member; and
the revision attachment feature comprises a revision translation coupler configured to removably mate with the prosthesis translation member.

11. The knee arthroplasty system of claim 7, wherein:
the prosthesis attachment feature comprises a prosthesis translation coupler on the bone-facing side of the knee arthroplasty prosthesis;
the primary attachment feature comprises a primary translation member configured to removably mate with the prosthesis translation coupler; and
the revision attachment feature comprises a revision translation member configured to removably mate with the prosthesis translation coupler.

12. A knee arthroplasty system for replacing a natural articular surface on a bone of a knee joint, the knee arthroplasty system comprising:
a knee arthroplasty prosthesis comprising:
a joint-facing side comprising an articular surface; and
a bone-facing side comprising a prosthesis attachment feature formed thereon;
a primary bone anchoring component comprising:
a primary bone engagement surface securable to a resected primary surface of the bone; and
a primary attachment feature; and
a revision bone anchoring component comprising:
a revision bone engagement surface securable to a resected revision surface of the bone; and
a revision attachment feature;
wherein:
the prosthesis attachment feature is configured to mate with either of the primary attachment feature and the revision attachment feature to couple either of the primary bone anchoring component and the revision bone anchoring component to the bone-facing side of the knee arthroplasty prosthesis; and
the prosthesis attachment feature is configured to rotatably and/or translatably mate with at least one of the primary attachment feature and the revision attachment feature.

13. The knee arthroplasty system of claim 12, wherein:
the prosthesis attachment feature comprises a prosthesis plate coupled to the bone-facing side of the knee arthroplasty prosthesis;
the primary attachment feature comprises a primary recess formed in the primary bone anchoring component and configured to rotatably mate with the prosthesis plate; and
the revision attachment feature comprises a revision recess formed in the revision bone anchoring component and configured to rotatably mate with the prosthesis plate.

14. The knee arthroplasty system of claim 12, wherein:
the prosthesis attachment feature comprises a prosthesis recess formed in the bone-facing side of the knee arthroplasty prosthesis;
the primary attachment feature comprises a primary plate coupled to the primary bone anchoring component and configured to rotatably mate with the prosthesis recess; and
the revision attachment feature comprises a revision plate coupled to the revision bone anchoring component and configured to rotatably mate with the prosthesis recess.

15. The knee arthroplasty system of claim 12, wherein:
the prosthesis attachment feature comprises a prosthesis translation member on the bone-facing side of the knee arthroplasty prosthesis;
the primary attachment feature comprises a primary translation coupler configured to translatably mate with the prosthesis translation member; and
the revision attachment feature comprises a revision translation coupler configured to translatably mate with the prosthesis translation member.

16. The knee arthroplasty system of claim 12, wherein:
the prosthesis attachment feature comprises a prosthesis translation coupler on the bone-facing side of the knee arthroplasty prosthesis;
the primary attachment feature comprises a primary translation member configured to translatably mate with the prosthesis translation coupler; and
the revision attachment feature comprises a revision translation member configured to translatably mate with the prosthesis translation coupler.

17. The knee arthroplasty system of claim 12, wherein:
the prosthesis attachment feature comprises a prosthesis recess formed in the bone-facing side of the knee arthroplasty prosthesis; and
the primary attachment feature comprises a primary plate coupled to the primary bone anchoring component and configured to rotatably mate with the prosthesis recess to rotatably couple the primary bone anchoring component to the bone-facing side of the knee arthroplasty prosthesis;
wherein:
the primary plate further comprises a primary translation member coupled to the primary plate; and
the primary bone anchoring component further comprises a primary translation coupler configured to translatably mate with the primary translation member and enable the primary bone anchoring component to:
translate along the primary translation member in a first direction with respect to the primary plate; and
translate along the primary translation member in a second direction, opposite the first direction, with respect to the primary plate.

18. The knee arthroplasty system of claim 12, wherein:
the prosthesis attachment feature comprises a prosthesis recess formed in the bone-facing side of the knee arthroplasty prosthesis; and
the revision attachment feature comprises a revision plate coupled to the revision bone anchoring component and configured to rotatably mate with the prosthesis recess to rotatably couple the revision bone anchoring component to the bone-facing side of the knee arthroplasty prosthesis;
wherein:
the revision plate further comprises a revision translation member coupled to the revision plate; and
the revision bone anchoring component further comprises a revision translation coupler configured to translatably mate with the revision translation member and enable the revision bone anchoring component to:
translate along the revision translation member in a first direction with respect to the revision plate; and
translate along the revision translation member in a second direction, opposite the first direction, with respect to the revision plate.

19. The knee arthroplasty system of claim 12, wherein at least a majority of the primary bone anchoring component is located outside an intramedullary canal of the bone when the primary bone anchoring component is anchored to the bone.

20. The knee arthroplasty system of claim 12, wherein the revision bone anchoring component comprises a stem, wherein a distal portion of the stem is securable within an intramedullary canal of the bone when the revision bone anchoring component is anchored to the bone.

21. An arthroplasty system for replacing a natural articular surface on a bone, the arthroplasty system comprising:
an arthroplasty prosthesis comprising:
a joint-facing side comprising an articular surface; and
a bone-facing side comprising a prosthesis attachment feature; and
a bone anchoring component comprising:
a bone engagement surface securable to a resected surface of the bone; and
a component attachment feature;
wherein:
the component attachment feature is configured to removably mate with the prosthesis attachment feature to removably couple the bone anchoring component to the bone-facing side of the arthroplasty prosthesis; and
the prosthesis attachment feature comprises a circular shape having a diameter of at least 20 mm.

22. The arthroplasty system of claim 21, wherein:
the prosthesis attachment feature comprises a first circular shape having a first diameter of at least 20 mm;
the component attachment feature comprises a second circular shape having a second diameter of at least 20 mm; and
the prosthesis attachment feature and the component attachment feature comprise complementary circular shapes with each other.

23. The arthroplasty system of claim 21, wherein:
the prosthesis attachment feature comprises a prosthesis plate coupled to the bone-facing side of the arthroplasty prosthesis; and
the component attachment feature comprises a component recess configured to removably mate with the prosthesis plate to removably couple the bone anchoring component to the bone-facing side of the arthroplasty prosthesis.

24. The arthroplasty system of claim 21, wherein:
the prosthesis attachment feature comprises a prosthesis recess formed in the bone-facing side of the arthroplasty prosthesis; and
the component attachment feature comprises a component plate coupled to the bone anchoring component and configured to removably mate with the prosthesis recess to removably couple the bone anchoring component to the bone-facing side of the arthroplasty prosthesis.

25. The arthroplasty system of claim 21, wherein:
the arthroplasty prosthesis comprises a tibial base plate comprising a tibial articular surface on the joint-facing side of the tibial base plate;
the tibial base plate comprises at least one first anti-rotation feature formed on the bone-facing side of the tibial base plate; and
the bone anchoring component comprises at least one second anti-rotation feature configured to removably mate with the first anti-rotation feature and resist rotation of the bone anchoring component with respect to the tibial base plate.
